SEQUENCE LISTING (23 sequences)

SEQ ID NO: 1 - DNA, 8187 bp, Artificial Sequence
  Cloning vector pMARES-neo (MCS)
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagtctactatgtagatatctgctactacctgtattatgcctctacctttattagcagagttatctgtactgttggcatgacaatcatttgttaatatgacttgcctttcctttttctgctattcttgatcaaatggctcctctttcttgctcctctcatttctcctgccttcacttggacgtgcttcacgtagtctgtgcttatgactggattaaaaattgatatggacttatcctaatgttgttcgtcataatatgggttttatggtccattattatttcctatgcattgatctggagaaggcttcaatccttttactctttgtggaaaatatctgtaaaccttctggttcactctgctatagcaatttcagtttaggctagtaagcatgaggatgcctccttctctgatttttcccacagtctgttggtcacagaataacctgagtgattactgatgaaagagtgagaatgttattgatagtcacaatgacaaaaaacaaacaactacagtcaaaatgtttctctttttattagtggattatatttcctgacctatatctggcaggactctttagagaggtagctgaagctgctgttatgaccactagagggaagaagatacctgtggagctaatggtccaagatggtggagccccaagcaaggaagttgttaaggagcccttttgattgaaggtgggtgcccccaccttacagggacaggacatctggatactcctcccagtttctccagtttccctttttcctaatatatctcctgataaaatgtctatactcacttccccatttctaataataaagcaaaggctagttagtaagacatcaccttgcattttgaaaatgccatagactttcaaaattatttcatacatcggtctttctttatttcaagagtccagaaatggcaacattacctttgattcaatgtaatggaaagagctctttcaagagacagagaaaagaataatttaatttctttccccacacctccttccctgtctcttaccctatcttccttccttctaccctccccatttctctctctcatttctcagaagtatattttgaaaggattcatagcagacagctaaggctggttttttctaagtgaagaagtgatattgagaaggtagggttgcatgagccctttcagttttttagtttatatacatctgtattgttagaatgttttataatataaataaaattatttctcagttatatactagctatgtaacctgtggatatttccttaagtattacaagctatacttaactcacttggaaaactcaaataaatacctgcttcatagttattaataaggattaagtgagataatgcccataagattcctattaataacagataaatacatacacacacacacacattgaaaggattcttactttgtgctaggaactataataagttcattgatgcattatatcattaagttctaatttcaacactagaaggcaggtattatctaaatttcatactggatacctccaaactcataaagataattaaattgccttttgtcatatatttattcaaaagggtaaactcaaactatggcttgtctaattttatatatcaccctactgaacatgaccctattgtgatattttataaaattattctcaagttattatgaggatgttgaaagacagagaggatggggtgctatgccccaaatcagcctcacaattaagctaagcagctaagagtcttgcagggtagtgtagggaccacagggttaagggggcagtagaattatactcccactttagtttcatttcaaacaatccatacacacacagccctgagcacttacaaattatactacgctctatactttttgtttaaatgtataaataagtggatgaaagaatagatagatagacagatagatgatagatagaataaatgcttgccttcatagctgtctccctaccttgttcaaaatgttcctgtccagaccaaagtaccttgccttcacttaagtaatcaattcctaggttatattctgatgtcaaaggaagtcaaaagatgtgaaaaacaatttctgacccacaactcatgctttgtagatgactagatcaaaaaatttcagccatatcttaacagtgagtgaacaggaaatctcctcttttccctacatctgagatcccagcttctaagaccttcaatactcactcttgatgcaacagaccttggaagtatacaggagagctgaacttggtcaacaaaggagaaaagtttgttggcctccaaaggcacagctcaaacttttcaagccttctctaatcttaaaggtaaacaagggtctcatttctttgagaacttcagggaaaatagacaaggacttgcctggtgcttttggtaggggagcttgcactttccccctttctggaggaaatatttatccccaggtagttccctttttgcaccagtggttctttgaagagacttccacctgggaacagttaaacagcaactacagggccttgaactgcacactttcagtccggtcctcacagttgaaaagacctaagcttgtgcctgatttaagcctttttggtcataaaacattcaattctaatctccctctcaaccctacagtcacccatttggtatattaaagatgtgttgtctactgtctagtatccctcaagtagtgtcaggaattagtcatttaaatagtctgcaagccaggagtggtggctcatgtctgtaattccagcactggagaggtagaagtgggaggactgcttgagctcaagagtttgatattatcctggacaacatagcaagacctcgtctctacttaaaaaaaaaaaaattagccaggcatgtgatgtacacctgtagtcccagctactcaggaggccgaaatgggaactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttggtaccgagctcggatcgatatctgcggcctagctagcgcttaaggcctgttaaccggtcgtacgtctccggattcgaattcggatccgcggccgcatagataactgatccagtgtgctggaattaattcgctgtctgcgagggccagctgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttgaggtgtggcaggcttgagatctggccatacacttgagtgacaatgacatccactttgcctttctctccacaggtgtccactcccaggtccaactgcaggtcgagcatgcatctagggcggccaattccgcccctctccctcccccccccctaacgttactggccgaagccgcttggaataaggccggtgtgcgtttgtctatatgtgattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgccacaacccgggataattcctgcagccaatatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatcaattctctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgtcgagtgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 2 - DNA, 1082 bp, Artificial Sequence
  Insert hygroR
cccgggataattcctgcagccaatatggatagatccggaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagtctgaggggatcaattctctaga

SEQ ID NO: 3 - protein, 2136 aa, Artificial Sequence
  Amino acid sequence of D1D2D`D3-HSA-TER119scFv (ID 3260)
MIPARFAGVLLALALILPGTLCAEGTRGRSSTARCSLFGSDFVNTFDGSMYSFAGYCSYLLAGGCQKRSFSIIGDFQNGKRVSLSVYLGEFFDIHLFVNGTVTQGDQRVSMPYASKGLYLETEAGYYKLSGEAYGFVARIDGSGNFQVLLSDRYFNKTCGLCGNFNIFAEDDFMTQEGTLTSDPYDFANSWALSSGEQWCERASPPSSSCNISSGEMQKGLWEQCQLLKSTSVFARCHPLVDPEPFVALCEKTLCECAGGLECACPALLEYARTCAQEGMVLYGWTDHSACSPVCPAGMEYRQCVSPCARTCQSLHINEMCQERCVDGCSCPEGQLLDEGLCVESTECPCVHSGKRYPPGTSLSRDCNTCICRNSQWICSNEECPGECLVTGQSHFKSFDNRYFTFSGICQYLLARDCQDHSFSIVIETVQCADDRDAVCTRSVTVRLPGLHNSLVKLKHGAGVAMDGQDVQLPLLKGDLRIQHTVTASVRLSYGEDLQMDWDGRGRLLVKLSPVYAGKTCGLCGNYNGNQGDDFLTPSGLAEPRVEDFGNAWKLHGDCQDLQKQHSDPCALNPRMTRFSEEACAVLTSPTFEACHRAVSPLPYLRNCRYDVCSCSDGRECLCGALASYAAACAGRGVRVAWREPGRCELNCPKGQVYLQCGTPCNLTCRSLSYPDEECNEACLEGCFCPPGLYMDERGDCVPKAQCPCYYDGEIFQPEDIFSDHHTMCYCEDGFMHCTMSGVPGSLLPDAVLSSPLSHRSKRSLSCRPPMVKLVCPADNLRAEGLECTKTCQNYDLECMSMGCVSGCLCPPGMVRHENRCVALERCPCFHQGKEYAPGETVKIGCNTCVCRDRKWNCTDHVCDATCSTIGMAHYLTFDGLKYLFPGECQYVLVQDYCGSNPGTFRILVGNKGCSHPSVKCKKRVTILVEGGEIELFDGEVNVKRPMKDETHFEVVESGRYIILLLGKALSVVWDRHLSISVVLKQTYQEKVCGLCGNFDGIQNNDLTSSNLQVEEDPVDFGNSWKVSSQCADTRKVPLDSSPATCHNNIMKQTMVDSSCRILTSDVFQDCNKLVDPEPYLDVCIYDTCSCESIGDCACFCDTIAAYAHVCAQHGKVVTWRTATLCPQSCEERNLRENGYECEWRYNSCAPACQVTCQHPEPLACPVQCVEGCHAHCPPGKILDELLQTCVDPEDCPVCEVAGRRFASGKKVTLNPSDPEHCQICHCDVVNLTCEACQEPGGSSGGSGGSGGSGGSGGSGGSGGSGGSGGSGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLTGGSGGSGGSGGSGGSGGSGGSGGSGGSGGSEVKLQESGGGLVQPGGSLKLSCVASGFTFRDHWMNWVRQAPGKTMEWIGDIRPDGSDTNYAPSVRNRFTISRDNARSILYLQMSNMRSDYTATYYCVRDSPTRAGLMDAWGQGTSVTVSSGGGGSGGGGSGGGGSGGGGSDIQMTQSPSVLSASVGDRVTLNCKASQNINKYLNWYQQKLGEAPKVLIYNTNNLQTGIPSRFSGSGSGTDFTLTISSLQPEDFATYFCFQHYTWPTFGGGTKLEIKR

SEQ ID NO: 4 - DNA, 6434 bp, Artificial Sequence
  DNA sequence encoding D1D2D`D3-HSA-TER119scFv (ID 3260)
gctagccaccatgattcctgccagatttgccggggtgctgcttgctctggccctcattttgccagggaccctttgtgcagaaggaactcgcggcaggtcatccacggcccgatgcagccttttcggaagtgacttcgtcaacacctttgatgggagcatgtacagctttgcgggatactgcagttacctcctggcagggggctgccagaaacgctccttctcgattattggggacttccagaatggcaagagagtgagcctctccgtgtatcttggggaattttttgacatccatttgtttgtcaatggtaccgtgacacagggggaccaaagagtctccatgccctatgcctccaaagggctgtatctagaaactgaggctgggtactacaagctgtccggtgaggcctatggctttgtggccaggatcgatggcagcggcaactttcaagtcctgctgtcagacagatacttcaacaagacctgcgggctgtgtggcaactttaacatctttgctgaagatgactttatgacccaagaagggaccttgacctcggacccttatgactttgccaactcatgggctctgagcagtggagaacagtggtgtgaacgggcatctcctcccagcagctcatgcaacatctcctctggggaaatgcagaagggcctgtgggagcagtgccagcttctgaagagcacctcggtgtttgcccgctgccaccctctggtggaccccgagccttttgtggccctgtgtgagaagactttgtgtgagtgtgctggggggctggagtgcgcctgccctgccctcctggagtacgcccggacctgtgcccaggagggaatggtgctgtacggctggaccgaccacagcgcgtgcagcccagtgtgccctgctggtatggagtataggcagtgtgtgtccccttgcgccaggacctgccagagcctgcacatcaatgaaatgtgtcaggagcgatgcgtggatggctgcagctgccctgagggacagctcctggatgaaggcctctgcgtggagagcaccgagtgtccctgcgtgcattccggaaagcgctaccctcccggcacctccctctctcgagactgcaacacctgcatttgccgaaacagccagtggatctgcagcaatgaagaatgtccaggggagtgccttgtcacaggtcaatcacacttcaagagctttgacaacagatacttcaccttcagtgggatctgccagtacctgctggcccgggattgccaggaccactccttctccattgtcattgagactgtccagtgtgctgatgaccgcgacgctgtgtgcacccgctccgtcaccgtccggctgcctggcctgcacaacagccttgtgaaactgaagcatggggcaggagttgccatggatggccaggacgtccagctccccctcctgaaaggtgacctccgcatccagcatacagtgacggcctccgtgcgcctcagctacggggaggacctgcagatggactgggatggccgcgggaggctgctggtgaagctgtcccccgtctatgccgggaagacctgcggcctgtgtgggaattacaatggcaaccagggcgacgacttccttaccccctctgggctggcggagccccgggtggaggacttcgggaacgcctggaagctgcacggggactgccaggacctgcagaagcagcacagcgatccctgcgccctcaacccgcgcatgaccaggttctccgaggaggcgtgcgcggtcctgacgtcccccacattcgaggcctgccatcgtgccgtcagcccgctgccctacctgcggaactgccgctacgacgtgtgctcctgctcggacggccgcgagtgcctgtgcggcgccctggccagctatgccgcggcctgcgcggggagaggcgtgcgcgtcgcgtggcgcgagccaggccgctgtgagctgaactgcccgaaaggccaggtgtacctgcagtgcgggaccccctgcaacctgacctgccgctctctctcttacccggatgaggaatgcaatgaggcctgcctggagggctgcttctgccccccagggctctacatggatgagaggggggactgcgtgcccaaggcccagtgcccctgttactatgacggtgagatcttccagccagaagacatcttctcagaccatcacaccatgtgctactgtgaggatggcttcatgcactgtaccatgagtggagtccccggaagcttgctgcctgacgctgtcctcagcagtcccctgtctcatcgcagcaaaaggagcctatcctgtcggccccccatggtcaagctggtgtgtcccgctgacaacctgcgggctgaagggctcgagtgtaccaaaacgtgccagaactatgacctggagtgcatgagcatgggctgtgtctctggctgcctctgccccccgggcatggtccggcatgagaacagatgtgtggccctggaaaggtgtccctgcttccatcagggcaaggagtatgcccctggagaaacagtgaagattggctgcaacacttgtgtctgtcgggaccggaagtggaactgcacagaccatgtgtgtgatgccacgtgctccacgatcggcatggcccactacctcaccttcgacgggctcaaatacctgttccccggggagtgccagtacgttctggtgcaggattactgcggcagtaaccctgggacctttcggatcctagtggggaataagggatgcagccacccctcagtgaaatgcaagaaacgggtcaccatcctggtggagggaggagagattgagctgtttgacggggaggtgaatgtgaagaggcccatgaaggatgagactcactttgaggtggtggagtctggccggtacatcattctgctgctgggcaaagccctctccgtggtctgggaccgccacctgagcatctccgtggtcctgaagcagacataccaggagaaagtgtgtggcctgtgtgggaattttgatggcatccagaacaatgacctcaccagcagcaacctccaagtggaggaagaccctgtggactttgggaactcctggaaagtgagctcgcagtgtgctgacaccagaaaagtgcctctggactcatcccctgccacctgccataacaacatcatgaagcagacgatggtggattcctcctgtagaatccttaccagtgacgtcttccaggactgcaacaagctggtggaccccgagccatatctggatgtctgcatttacgacacctgctcctgtgagtccattggggactgcgcctgcttctgcgacaccattgctgcctatgcccacgtgtgtgcccagcatggcaaggtggtgacctggaggacggccacattgtgcccccagagctgcgaggagaggaatctccgggagaacgggtatgagtgtgagtggcgctataacagctgtgcacctgcctgtcaagtcacgtgtcagcaccctgagccactggcctgccctgtgcagtgtgtggagggctgccatgcccactgccctccagggaaaatcctggatgagcttttgcagacctgcgttgaccctgaagactgtccagtgtgtgaggtggctggccggcgttttgcctcaggaaagaaagtcaccttgaatcccagtgaccctgagcactgccagatttgccactgtgatgttgtcaacctcacctgtgaagcctgccaggagccgggaggctcgagcgggggatctggcgggtctggaggctctggagggtcgggaggctctggaggctctgggggatctggcgggtctggagggtcgggatccgatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagctcgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttaaccggtggcagcggtgggtccggtggcagtggaggctctggcggctcaggtgggtccggaggaagcggtgggagtggagggtctggaggctccgaggtgaagctgcaggaatccggaggtggcctggtacagccaggtggcagcctgaagttgagctgcgtagcatccggatttaccttccgggaccattggatgaattgggtgagacaagctccaggaaagaccatggagtggattggcgacatcagacccgacggcagcgataccaactatgccccatcagtgcgaaatcgcttcaccatttccagggataatgctcgctctatcctgtatcttcagatgagcaacatgcggtcagactatacagccacttactactgtgtgcgcgatagtcccacacgtgcagggttgatggatgcctggggtcaagggacgtccgtcactgtgagttctggcggcggagggagtggaggtggcgggagtggtggaggtggcagtggtggcggcggctccgacatacagatgactcagtcacctagcgttcttagcgcttccgttggggatcgtgtcaccctcaattgtaaggcttcccagaacatcaacaagtacctgaactggtaccagcagaaactcggagaagcacccaaagtgctgatctacaacacgaacaatctccagactggtattccctctaggttttctgggtccggaagcgggacagacttcacactgactatttccagtctgcaacctgaggattttgccacctacttctgctttcagcactatacctggcctacatttggcggaggtacaaagctggagatcaaacggtagtgatagcggccgc

SEQ ID NO: 5 - protein, 1282 aa, Artificial Sequence
  Amino acid sequence of D1D2D`D3 8His-tag (ID 3748)
MIPARFAGVLLALALILPGTLCAEGTRGRSSTARCSLFGSDFVNTFDGSMYSFAGYCSYLLAGGCQKRSFSIIGDFQNGKRVSLSVYLGEFFDIHLFVNGTVTQGDQRVSMPYASKGLYLETEAGYYKLSGEAYGFVARIDGSGNFQVLLSDRYFNKTCGLCGNFNIFAEDDFMTQEGTLTSDPYDFANSWALSSGEQWCERASPPSSSCNISSGEMQKGLWEQCQLLKSTSVFARCHPLVDPEPFVALCEKTLCECAGGLECACPALLEYARTCAQEGMVLYGWTDHSACSPVCPAGMEYRQCVSPCARTCQSLHINEMCQERCVDGCSCPEGQLLDEGLCVESTECPCVHSGKRYPPGTSLSRDCNTCICRNSQWICSNEECPGECLVTGQSHFKSFDNRYFTFSGICQYLLARDCQDHSFSIVIETVQCADDRDAVCTRSVTVRLPGLHNSLVKLKHGAGVAMDGQDVQLPLLKGDLRIQHTVTASVRLSYGEDLQMDWDGRGRLLVKLSPVYAGKTCGLCGNYNGNQGDDFLTPSGLAEPRVEDFGNAWKLHGDCQDLQKQHSDPCALNPRMTRFSEEACAVLTSPTFEACHRAVSPLPYLRNCRYDVCSCSDGRECLCGALASYAAACAGRGVRVAWREPGRCELNCPKGQVYLQCGTPCNLTCRSLSYPDEECNEACLEGCFCPPGLYMDERGDCVPKAQCPCYYDGEIFQPEDIFSDHHTMCYCEDGFMHCTMSGVPGSLLPDAVLSSPLSHRSKRSLSCRPPMVKLVCPADNLRAEGLECTKTCQNYDLECMSMGCVSGCLCPPGMVRHENRCVALERCPCFHQGKEYAPGETVKIGCNTCVCRDRKWNCTDHVCDATCSTIGMAHYLTFDGLKYLFPGECQYVLVQDYCGSNPGTFRILVGNKGCSHPSVKCKKRVTILVEGGEIELFDGEVNVKRPMKDETHFEVVESGRYIILLLGKALSVVWDRHLSISVVLKQTYQEKVCGLCGNFDGIQNNDLTSSNLQVEEDPVDFGNSWKVSSQCADTRKVPLDSSPATCHNNIMKQTMVDSSCRILTSDVFQDCNKLVDPEPYLDVCIYDTCSCESIGDCACFCDTIAAYAHVCAQHGKVVTWRTATLCPQSCEERNLRENGYECEWRYNSCAPACQVTCQHPEPLACPVQCVEGCHAHCPPGKILDELLQTCVDPEDCPVCEVAGRRFASGKKVTLNPSDPEHCQICHCDVVNLTCEACQEPGGSSGGSGGSGGSGGSGGSGGSGGSGGSGGSGGSHHHHHHHH

SEQ ID NO: 6 - DNA, 3869 bp, Artificial Sequence
  DNA sequence encoding D1D2D`D3 8His-tag (ID 3748)
gctagccaccatgatccccgctcgcttcgccggagtgctgctggccctggctctgatcctgcctggcaccctgtgtgccgagggcaccagaggcagatcctctaccgcccggtgctccctgttcggctccgatttcgtgaataccttcgacggctctatgtactccttcgccggctactgctcctacctgctggctggcggctgtcagaagcggtccttctccatcatcggcgattttcagaacggcaagcgggtgtccctgtccgtgtacctgggcgagttcttcgacatccacctgttcgtgaacggcaccgtgacccagggcgatcagagggtgtccatgccctacgccagcaagggcctgtacctggaaaccgaggccggctattataagctgagcggcgaggcttacggcttcgtggctagaatcgacggctccggcaacttccaggtgctgctgagcgaccggtactttaacaagacatgtggcctgtgcggaaacttcaacatcttcgccgaggacgatttcatgacccaggaaggcaccctgacaagcgacccctacgacttcgccaacagctgggccctgtcctccggcgagcagtggtgcgagagagccagcccaccctccagctcttgtaacatctctagcggcgagatgcagaaaggactgtgggaacagtgtcagctgctgaaatccacctccgtgttcgccagatgtcatccactggtggaccctgaacccttcgtcgctctgtgcgaaaagaccctgtgcgagtgcgctggcggcctggaatgtgcctgtcccgctctgctggaatacgccagaacctgcgctcaggaaggcatggtcctgtacggatggacagatcactccgcctgctcccccgtgtgtcctgccggcatggaataccgccagtgcgtgtccccctgtgctcggacctgtcagtctctgcatatcaacgagatgtgccaggaacgctgcgtcgacggctgctcttgtcccgagggccagctgctggacgagggcctgtgtgtcgagtccaccgagtgcccttgtgtgcacagcggcaagagatacccccctggcaccagcctgagccgggactgtaacacctgtatctgcagaaactctcagtggatctgttccaacgaggaatgccctggcgagtgtctggtcaccggccagtcccacttcaagtccttcgataaccggtacttcaccttttccggcatctgtcagtatctgctggctagagactgccaggatcacagcttctccatcgtgatcgagacagtgcagtgcgccgacgacagggacgccgtgtgcaccagatctgtgacagtgcggctgcccggactgcataactccctggtcaagctgaagcacggcgctggcgtggcaatggacggccaggatgtgcagctgcctctgctgaagggcgacctgagaatccagcacaccgtgaccgccagcgtgcggctgtcttacggcgaagatctgcagatggattgggacggcagaggccgcctgctggtcaaactgagccccgtgtacgctggcaagacttgcggactgtgcgggaactataacggcaatcagggggatgatttcctgaccccttccggcctggccgagcccagagtggaagattttggcaatgcttggaagctgcatggcgattgtcaggatctgcagaagcagcattccgacccctgtgccctgaaccccagaatgacccggttcagcgaagaggcctgcgctgtgctgaccagccctaccttcgaggcctgtcacagagccgtgtcccccctgccttacctgagaaactgcagatacgatgtgtgctcttgcagcgacggcagagaatgtctgtgtggcgctctggcctcctacgctgccgcttgtgctggaagaggcgtccgggtggcatggcgggaacctggcagatgcgagctgaattgtcccaagggacaggtctacctgcagtgtggcacaccttgtaatctgacctgccggtccctgtcctaccccgacgaagagtgcaacgaggcttgtctggaaggctgtttttgccctcccggcctgtacatggacgagcgcggcgattgtgtgcctaaggctcagtgtccttgctactacgatggcgagatctttcagcccgaggatatcttcagcgatcaccacaccatgtgttattgcgaggacggttttatgcactgcaccatgtccggcgtgcccggctccctgctgccagatgctgtgctgtcctccccactgtcccaccggtccaagcggtccctgagctgccggcctcctatggtcaaactggtctgccctgccgataacctgcgggccgagggactggaatgcaccaagacctgtcagaactacgatctggaatgcatgtctatgggctgcgtgtccggctgtctgtgtcctcccggaatggtccgacacgagaatcgctgcgtggcactggaacggtgcccatgttttcaccaaggcaaagagtacgctcccggcgagactgtgaagatcggctgtaatacctgcgtgtgccgggacagaaagtggaattgcaccgatcacgtgtgcgacgccacctgtagcaccatcggcatggctcattatctgacctttgatggcctgaagtacctgtttccaggcgaatgccagtatgtgctggtccaggactattgtggctccaaccccggcaccttcagaatcctggtcggaaacaagggctgctcccacccttccgtgaagtgtaaaaagagagtgaccattctggtcgagggcggcgagatcgaactgttcgacggcgaagtgaacgtcaaacggcctatgaaggacgaaacccatttcgaggtggtcgagagcggccggtatattatcctgctcctggggaaggctctgagcgtcgtgtgggaccggcacctgtccattagtgtggtgctgaaacagacctatcaggaaaaagtctgcgggctctgcggcaatttcgacggaatccagaacaacgacctgacctctagcaacctgcaggtcgaagaggaccccgtggacttcggcaatagctggaaggtgtcctcccagtgtgccgacacccggaaggtccccctggacagctctcccgccacctgtcacaacaatattatgaagcagacaatggtcgactccagctgcagaatcctgacctccgacgtgttccaggattgcaacaaactggtcgatcctgaaccttacctggacgtgtgtatctacgacacctgtagctgcgagagcatcggcgactgtgcctgtttctgtgacactatcgccgcttacgcccatgtgtgcgctcagcacggaaaggtcgtgacatggcgcaccgccaccctgtgtcctcagtcctgcgaggaacggaacctgcgcgagaatggctacgagtgcgagtggcggtacaactcctgcgcccctgcctgccaggtcacatgccagcatcccgagcccctggcttgtcccgtccagtgcgtggaaggatgtcacgcccactgtccccctggcaagattctggacgagctgctgcagacatgtgtggacccagaggactgccccgtgtgcgaagtggccggcagaagattcgcctccggcaagaaagtgacactgaacccctccgaccccgagcattgtcagatctgtcactgcgacgtggtcaacctgacatgcgaggcttgccaggaaccaggcggatcctctggcggcagcggcggaagtggcggttctggcggatcaggcggcagcggcggaagcggtggaagcggaggatctggtggctccggcggctctcatcaccaccatcatcaccaccactgatagcggccgc

SEQ ID NO: 7 - protein, 2136 aa, Artificial Sequence
  Amino acid sequence of D1D2D`D3(EYA)-HSA- TER119scFv (ID 3794)
MIPARFAGVLLALALILPGTLCAEGTRGRSSTARCSLFGSDFVNTFDGSMYSFAGYCSYLLAGGCQKRSFSIIGDFQNGKRVSLSVYLGEFFDIHLFVNGTVTQGDQRVSMPYASKGLYLETEAGYYKLSGEAYGFVARIDGSGNFQVLLSDRYFNKTCGLCGNFNIFAEDDFMTQEGTLTSDPYDFANSWALSSGEQWCERASPPSSSCNISSGEMQKGLWEQCQLLKSTSVFARCHPLVDPEPFVALCEKTLCECAGGLECACPALLEYARTCAQEGMVLYGWTDHSACSPVCPAGMEYRQCVSPCARTCQSLHINEMCQERCVDGCSCPEGQLLDEGLCVESTECPCVHSGKRYPPGTSLSRDCNTCICRNSQWICSNEECPGECLVTGQSHFKSFDNRYFTFSGICQYLLARDCQDHSFSIVIETVQCADDRDAVCTRSVTVRLPGLHNSLVKLKHGAGVAMDGQDVQLPLLKGDLRIQHTVTASVRLSYGEDLQMDWDGRGRLLVKLSPVYAGKTCGLCGNYNGNQGDDFLTPSGLAEPRVEDFGNAWKLHGDCQDLQKQHSDPCALNPRMTRFSEEACAVLTSPTFEACHRAVSPLPYLRNCRYDVCSCSDGRECLCGALASYAAACAGRGVRVAWREPGRCELNCPKGQVYLQCGTPCNLTCRSLSYPDEECNEACLEGCFCPPGLYMDERGDCVPKAQCPCYYDGEIFQPEDIFSDHHTMCYCEDGFMHCTMSGVPGSLLPDAVLSSPLSHRSKRELYCRPPMVKLVCPADNLRAEGLECTKTCQNYDLECMSMGCVSGCLCPPGMVRHENRCVALERCPCFHQGKEYAPGETVKIGCNTCVCRDRKWNCTDHVCDATCSTIGMAHYLTFDGLKYLFPGECQYVLVQDYCGSNPGTFRILVGNKGCSHPSVKCKKRVTILVEGGEIELFDGEVNVKRPMKDETHFEVVESGRYIILLLGKALSVVWDRHLSISVVLKQTYQEKVCGLCGNFDGIQNNDLTSSNLQVEEDPVDFGNSWKVSSQCADTRKVPLDSSPATCHNNIMKQTMVDSSCRILTSDVFQDCNKLVDPEPYLDACIYDTCSCESIGDCACFCDTIAAYAHVCAQHGKVVTWRTATLCPQSCEERNLRENGYECEWRYNSCAPACQVTCQHPEPLACPVQCVEGCHAHCPPGKILDELLQTCVDPEDCPVCEVAGRRFASGKKVTLNPSDPEHCQICHCDVVNLTCEACQEPGGSSGGSGGSGGSGGSGGSGGSGGSGGSGGSGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLTGGSGGSGGSGGSGGSGGSGGSGGSGGSGGSEVKLQESGGGLVQPGGSLKLSCVASGFTFRDHWMNWVRQAPGKTMEWIGDIRPDGSDTNYAPSVRNRFTISRDNARSILYLQMSNMRSDYTATYYCVRDSPTRAGLMDAWGQGTSVTVSSGGGGSGGGGSGGGGSGGGGSDIQMTQSPSVLSASVGDRVTLNCKASQNINKYLNWYQQKLGEAPKVLIYNTNNLQTGIPSRFSGSGSGTDFTLTISSLQPEDFATYFCFQHYTWPTFGGGTKLEIKR

SEQ ID NO: 8 - DNA, 6434 bp, Artificial Sequence
  DNA sequence encoding D1D2D`D3(EYA)-HSA- TER119scFv (ID 3794)
gctagccaccatgatccccgctcgcttcgccggagtgctgctggccctggctctgatcctgcctggcaccctgtgtgccgagggcaccagaggcagatcctctaccgcccggtgctccctgttcggctccgatttcgtgaataccttcgacggctctatgtactccttcgccggctactgctcctacctgctggctggcggctgtcagaagcggtccttctccatcatcggcgattttcagaacggcaagcgggtgtccctgtccgtgtacctgggcgagttcttcgacatccacctgttcgtgaacggcaccgtgacccagggcgatcagagggtgtccatgccctacgccagcaagggcctgtacctggaaaccgaggccggctattataagctgagcggcgaggcttacggcttcgtggctagaatcgacggctccggcaacttccaggtgctgctgagcgaccggtactttaacaagacatgtggcctgtgcggaaacttcaacatcttcgccgaggacgatttcatgacccaggaaggcaccctgacaagcgacccctacgacttcgccaacagctgggccctgtcctccggcgagcagtggtgcgagagagccagcccaccctccagctcttgtaacatctctagcggcgagatgcagaaaggactgtgggaacagtgtcagctgctgaaatccacctccgtgttcgccagatgtcatccactggtggaccctgaacccttcgtcgctctgtgcgaaaagaccctgtgcgagtgcgctggcggcctggaatgtgcctgtcccgctctgctggaatacgccagaacctgcgctcaggaaggcatggtcctgtacggatggacagatcactccgcctgctcccccgtgtgtcctgccggcatggaataccgccagtgcgtgtccccctgtgctcggacctgtcagtctctgcatatcaacgagatgtgccaggaacgctgcgtcgacggctgctcttgtcccgagggccagctgctggacgagggcctgtgtgtcgagtccaccgagtgcccttgtgtgcacagcggcaagagatacccccctggcaccagcctgagccgggactgtaacacctgtatctgcagaaactctcagtggatctgttccaacgaggaatgccctggcgagtgtctggtcaccggccagtcccacttcaagtccttcgataaccggtacttcaccttttccggcatctgtcagtatctgctggctagagactgccaggatcacagcttctccatcgtgatcgagacagtgcagtgcgccgacgacagggacgccgtgtgcaccagatctgtgacagtgcggctgcccggactgcataactccctggtcaagctgaagcacggcgctggcgtggcaatggacggccaggatgtgcagctgcctctgctgaagggcgacctgagaatccagcacaccgtgaccgccagcgtgcggctgtcttacggcgaagatctgcagatggattgggacggcagaggccgcctgctggtcaaactgagccccgtgtacgctggcaagacttgcggactgtgcgggaactataacggcaatcagggggatgatttcctgaccccttccggcctggccgagcccagagtggaagattttggcaatgcttggaagctgcatggcgattgtcaggatctgcagaagcagcattccgacccctgtgccctgaaccccagaatgacccggttcagcgaagaggcctgcgctgtgctgaccagccctaccttcgaggcctgtcacagagccgtgtcccccctgccttacctgagaaactgcagatacgatgtgtgctcttgcagcgacggcagagaatgtctgtgtggcgctctggcctcctacgctgccgcttgtgctggaagaggcgtccgggtggcatggcgggaacctggcagatgcgagctgaattgtcccaagggacaggtctacctgcagtgtggcacaccttgtaatctgacctgccggtccctgtcctaccccgacgaagagtgcaacgaggcttgtctggaaggctgtttttgccctcccggcctgtacatggacgagcgcggcgattgtgtgcctaaggctcagtgtccttgctactacgatggcgagatctttcagcccgaggatatcttcagcgatcaccacaccatgtgttattgcgaggacggttttatgcactgcaccatgtccggcgtgcccggctccctgctgccagatgctgtgctgtcctccccactgtcccaccggtccaagcgggagctgtactgccggcctcctatggtcaaactggtctgccctgccgataacctgcgggccgagggactggaatgcaccaagacctgtcagaactacgatctggaatgcatgtctatgggctgcgtgtccggctgtctgtgtcctcccggaatggtccgacacgagaatcgctgcgtggcactggaacggtgcccatgttttcaccaaggcaaagagtacgctcccggcgagactgtgaagatcggctgtaatacctgcgtgtgccgggacagaaagtggaattgcaccgatcacgtgtgcgacgccacctgtagcaccatcggcatggctcattatctgacctttgatggcctgaagtacctgtttccaggcgaatgccagtatgtgctggtccaggactattgtggctccaaccccggcaccttcagaatcctggtcggaaacaagggctgctcccacccttccgtgaagtgtaaaaagagagtgaccattctggtcgagggcggcgagatcgaactgttcgacggcgaagtgaacgtcaaacggcctatgaaggacgaaacccatttcgaggtggtcgagagcggccggtatattatcctgctcctggggaaggctctgagcgtcgtgtgggaccggcacctgtccattagtgtggtgctgaaacagacctatcaggaaaaagtctgcgggctctgcggcaatttcgacggaatccagaacaacgacctgacctctagcaacctgcaggtcgaagaggaccccgtggacttcggcaatagctggaaggtgtcctcccagtgtgccgacacccggaaggtccccctggacagctctcccgccacctgtcacaacaatattatgaagcagacaatggtcgactccagctgcagaatcctgacctccgacgtgttccaggattgcaacaaactggtcgatcctgaaccttacctggacgcctgtatctacgacacctgtagctgcgagagcatcggcgactgtgcctgtttctgtgacactatcgccgcttacgcccatgtgtgcgctcagcacggaaaggtcgtgacatggcgcaccgccaccctgtgtcctcagtcctgcgaggaacggaacctgcgcgagaatggctacgagtgcgagtggcggtacaactcctgcgcccctgcctgccaggtcacatgccagcatcccgagcccctggcttgtcccgtccagtgcgtggaaggatgtcacgcccactgtccccctggcaagattctggacgagctgctgcagacatgtgtggacccagaggactgccccgtgtgcgaagtggccggcagaagattcgcctccggcaagaaagtgacactgaacccctccgaccccgagcattgtcagatctgtcactgcgacgtggtcaacctgacatgcgaggcttgccaggaaccaggcggatcctctggcggcagcggcggaagtggcggttctggcggatcaggcggaagcggtggaagcggaggatctggtggctccggcggctctggatccgatgcccacaagagcgaggtggcccaccggttcaaggacctgggagaggagaacttcaaagccctggtgctgatcgccttcgcccagtacctccagcagtgcccattcgaagaccatgtcaagctggtgaatgaggtcactgagtttgccaagacctgtgtggccgatgagtccgctgagaactgtgacaagagcctccacaccctcttcggagacaagctgtgcacagtggccaccctccgggagacctatggggaaatggctgactgctgtgcaaagcaggagcctgagaggaacgaatgcttcctgcagcacaaagatgacaaccccaacctcccccggctggtgcgcccagaggtggacgtgatgtgcactgccttccatgacaacgaggagaccttcctgaagaagtacctgtacgagattgcccgccgccacccttacttctatgcccccgaactcctcttctttgccaagagatacaaagccgccttcacagagtgctgccaggctgctgacaaagccgcctgcctgctgcccaagctcgatgagctccgggacgaagggaaggcctcctctgccaagcagcgcctcaagtgtgccagcctccaaaagtttggcgagcgggccttcaaagcctgggcagtggcccgcctgagccagcgcttccccaaagctgagtttgcagaggtgtccaagctggtgaccgacctcaccaaggtccacaccgaatgctgccatggcgacctgctggaatgtgccgatgacagggccgacctggccaaatacatctgtgagaaccaggactctatctccagcaagctgaaggaatgctgtgagaagcccctgctggagaagtcccactgcattgccgaagtggagaatgacgagatgcctgccgacctgcccagcctggctgccgacttcgtggaaagcaaggacgtgtgcaagaactacgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagctcgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttaaccggtggcagcggtgggtccggtggcagtggaggctctggcggctcaggtgggtccggaggaagcggtgggagtggagggtctggaggctccgaggtgaagctgcaggaatccggaggtggcctggtacagccaggtggcagcctgaagttgagctgcgtagcatccggatttaccttccgggaccattggatgaattgggtgagacaagctccaggaaagaccatggagtggattggcgacatcagacccgacggcagcgataccaactatgccccatcagtgcgaaatcgcttcaccatttccagggataatgctcgctctatcctgtatcttcagatgagcaacatgcggtcagactatacagccacttactactgtgtgcgcgatagtcccacacgtgcagggttgatggatgcctggggtcaagggacgtccgtcactgtgagttctggcggcggagggagtggaggtggcgggagtggtggaggtggcagtggtggcggcggctccgacatacagatgactcagtcacctagcgttcttagcgcttccgttggggatcgtgtcaccctcaattgtaaggcttcccagaacatcaacaagtacctgaactggtaccagcagaaactcggagaagcacccaaagtgctgatctacaacacgaacaatctccagactggtattccctctaggttttctgggtccggaagcgggacagacttcacactgactatttccagtctgcaacctgaggattttgccacctacttctgctttcagcactatacctggcctacatttggcggaggtacaaagctggagatcaaacggtagtgatagcggccgc

SEQ ID NO: 9 - protein, 1282 aa, Artificial Sequence
  Amino acid sequence of D1D2D`D3(EYA) 8His-tag (ID 3795)
MIPARFAGVLLALALILPGTLCAEGTRGRSSTARCSLFGSDFVNTFDGSMYSFAGYCSYLLAGGCQKRSFSIIGDFQNGKRVSLSVYLGEFFDIHLFVNGTVTQGDQRVSMPYASKGLYLETEAGYYKLSGEAYGFVARIDGSGNFQVLLSDRYFNKTCGLCGNFNIFAEDDFMTQEGTLTSDPYDFANSWALSSGEQWCERASPPSSSCNISSGEMQKGLWEQCQLLKSTSVFARCHPLVDPEPFVALCEKTLCECAGGLECACPALLEYARTCAQEGMVLYGWTDHSACSPVCPAGMEYRQCVSPCARTCQSLHINEMCQERCVDGCSCPEGQLLDEGLCVESTECPCVHSGKRYPPGTSLSRDCNTCICRNSQWICSNEECPGECLVTGQSHFKSFDNRYFTFSGICQYLLARDCQDHSFSIVIETVQCADDRDAVCTRSVTVRLPGLHNSLVKLKHGAGVAMDGQDVQLPLLKGDLRIQHTVTASVRLSYGEDLQMDWDGRGRLLVKLSPVYAGKTCGLCGNYNGNQGDDFLTPSGLAEPRVEDFGNAWKLHGDCQDLQKQHSDPCALNPRMTRFSEEACAVLTSPTFEACHRAVSPLPYLRNCRYDVCSCSDGRECLCGALASYAAACAGRGVRVAWREPGRCELNCPKGQVYLQCGTPCNLTCRSLSYPDEECNEACLEGCFCPPGLYMDERGDCVPKAQCPCYYDGEIFQPEDIFSDHHTMCYCEDGFMHCTMSGVPGSLLPDAVLSSPLSHRSKRELYCRPPMVKLVCPADNLRAEGLECTKTCQNYDLECMSMGCVSGCLCPPGMVRHENRCVALERCPCFHQGKEYAPGETVKIGCNTCVCRDRKWNCTDHVCDATCSTIGMAHYLTFDGLKYLFPGECQYVLVQDYCGSNPGTFRILVGNKGCSHPSVKCKKRVTILVEGGEIELFDGEVNVKRPMKDETHFEVVESGRYIILLLGKALSVVWDRHLSISVVLKQTYQEKVCGLCGNFDGIQNNDLTSSNLQVEEDPVDFGNSWKVSSQCADTRKVPLDSSPATCHNNIMKQTMVDSSCRILTSDVFQDCNKLVDPEPYLDACIYDTCSCESIGDCACFCDTIAAYAHVCAQHGKVVTWRTATLCPQSCEERNLRENGYECEWRYNSCAPACQVTCQHPEPLACPVQCVEGCHAHCPPGKILDELLQTCVDPEDCPVCEVAGRRFASGKKVTLNPSDPEHCQICHCDVVNLTCEACQEPGGSSGGSGGSGGSGGSGGSGGSGGSGGSGGSGGSHHHHHHHH

SEQ ID NO: 10 - DNA, 3869 bp, Artificial Sequence
  DNA sequence encoding D1D2D`D3(EYA) 8His-tag (ID 3795)
gctagccaccatgatccccgctcgcttcgccggagtgctgctggccctggctctgatcctgcctggcaccctgtgtgccgagggcaccagaggcagatcctctaccgcccggtgctccctgttcggctccgatttcgtgaataccttcgacggctctatgtactccttcgccggctactgctcctacctgctggctggcggctgtcagaagcggtccttctccatcatcggcgattttcagaacggcaagcgggtgtccctgtccgtgtacctgggcgagttcttcgacatccacctgttcgtgaacggcaccgtgacccagggcgatcagagggtgtccatgccctacgccagcaagggcctgtacctggaaaccgaggccggctattataagctgagcggcgaggcttacggcttcgtggctagaatcgacggctccggcaacttccaggtgctgctgagcgaccggtactttaacaagacatgtggcctgtgcggaaacttcaacatcttcgccgaggacgatttcatgacccaggaaggcaccctgacaagcgacccctacgacttcgccaacagctgggccctgtcctccggcgagcagtggtgcgagagagccagcccaccctccagctcttgtaacatctctagcggcgagatgcagaaaggactgtgggaacagtgtcagctgctgaaatccacctccgtgttcgccagatgtcatccactggtggaccctgaacccttcgtcgctctgtgcgaaaagaccctgtgcgagtgcgctggcggcctggaatgtgcctgtcccgctctgctggaatacgccagaacctgcgctcaggaaggcatggtcctgtacggatggacagatcactccgcctgctcccccgtgtgtcctgccggcatggaataccgccagtgcgtgtccccctgtgctcggacctgtcagtctctgcatatcaacgagatgtgccaggaacgctgcgtcgacggctgctcttgtcccgagggccagctgctggacgagggcctgtgtgtcgagtccaccgagtgcccttgtgtgcacagcggcaagagatacccccctggcaccagcctgagccgggactgtaacacctgtatctgcagaaactctcagtggatctgttccaacgaggaatgccctggcgagtgtctggtcaccggccagtcccacttcaagtccttcgataaccggtacttcaccttttccggcatctgtcagtatctgctggctagagactgccaggatcacagcttctccatcgtgatcgagacagtgcagtgcgccgacgacagggacgccgtgtgcaccagatctgtgacagtgcggctgcccggactgcataactccctggtcaagctgaagcacggcgctggcgtggcaatggacggccaggatgtgcagctgcctctgctgaagggcgacctgagaatccagcacaccgtgaccgccagcgtgcggctgtcttacggcgaagatctgcagatggattgggacggcagaggccgcctgctggtcaaactgagccccgtgtacgctggcaagacttgcggactgtgcgggaactataacggcaatcagggggatgatttcctgaccccttccggcctggccgagcccagagtggaagattttggcaatgcttggaagctgcatggcgattgtcaggatctgcagaagcagcattccgacccctgtgccctgaaccccagaatgacccggttcagcgaagaggcctgcgctgtgctgaccagccctaccttcgaggcctgtcacagagccgtgtcccccctgccttacctgagaaactgcagatacgatgtgtgctcttgcagcgacggcagagaatgtctgtgtggcgctctggcctcctacgctgccgcttgtgctggaagaggcgtccgggtggcatggcgggaacctggcagatgcgagctgaattgtcccaagggacaggtctacctgcagtgtggcacaccttgtaatctgacctgccggtccctgtcctaccccgacgaagagtgcaacgaggcttgtctggaaggctgtttttgccctcccggcctgtacatggacgagcgcggcgattgtgtgcctaaggctcagtgtccttgctactacgatggcgagatctttcagcccgaggatatcttcagcgatcaccacaccatgtgttattgcgaggacggttttatgcactgcaccatgtccggcgtgcccggctccctgctgccagatgctgtgctgtcctccccactgtcccaccggtccaagcgggagctgtactgccggcctcctatggtcaaactggtctgccctgccgataacctgcgggccgagggactggaatgcaccaagacctgtcagaactacgatctggaatgcatgtctatgggctgcgtgtccggctgtctgtgtcctcccggaatggtccgacacgagaatcgctgcgtggcactggaacggtgcccatgttttcaccaaggcaaagagtacgctcccggcgagactgtgaagatcggctgtaatacctgcgtgtgccgggacagaaagtggaattgcaccgatcacgtgtgcgacgccacctgtagcaccatcggcatggctcattatctgacctttgatggcctgaagtacctgtttccaggcgaatgccagtatgtgctggtccaggactattgtggctccaaccccggcaccttcagaatcctggtcggaaacaagggctgctcccacccttccgtgaagtgtaaaaagagagtgaccattctggtcgagggcggcgagatcgaactgttcgacggcgaagtgaacgtcaaacggcctatgaaggacgaaacccatttcgaggtggtcgagagcggccggtatattatcctgctcctggggaaggctctgagcgtcgtgtgggaccggcacctgtccattagtgtggtgctgaaacagacctatcaggaaaaagtctgcgggctctgcggcaatttcgacggaatccagaacaacgacctgacctctagcaacctgcaggtcgaagaggaccccgtggacttcggcaatagctggaaggtgtcctcccagtgtgccgacacccggaaggtccccctggacagctctcccgccacctgtcacaacaatattatgaagcagacaatggtcgactccagctgcagaatcctgacctccgacgtgttccaggattgcaacaaactggtcgatcctgaaccttacctggacgcctgtatctacgacacctgtagctgcgagagcatcggcgactgtgcctgtttctgtgacactatcgccgcttacgcccatgtgtgcgctcagcacggaaaggtcgtgacatggcgcaccgccaccctgtgtcctcagtcctgcgaggaacggaacctgcgcgagaatggctacgagtgcgagtggcggtacaactcctgcgcccctgcctgccaggtcacatgccagcatcccgagcccctggcttgtcccgtccagtgcgtggaaggatgtcacgcccactgtccccctggcaagattctggacgagctgctgcagacatgtgtggacccagaggactgccccgtgtgcgaagtggccggcagaagattcgcctccggcaagaaagtgacactgaacccctccgaccccgagcattgtcagatctgtcactgcgacgtggtcaacctgacatgcgaggcttgccaggaaccaggcggatcctctggcggcagcggcggaagtggcggttctggcggatcaggcggcagcggcggaagcggtggaagcggaggatctggtggctccggcggctctcatcaccaccatcatcaccaccactgatagcggccgc

SEQ ID NO: 11 - protein, 794 aa, Artificial Sequence
  Amino acid sequence of Furin (ID 863)
MELRPWLLWVVAATGTLVLLAADAQGQKVFTNTWAVRIPGGPAVANSVARKHGFLNLGQIFGDYYHFWHRGVTKRSLSPHRPRHSRLQREPQVQWLEQQVAKRRTKRDVYQEPTDPKFPQQWYLSGVTQRDLNVKAAWAQGYTGHGIVVSILDDGIEKNHPDLAGNYDPGASFDVNDQDPDPQPRYTQMNDNRHGTRCAGEVAAVANNGVCGVGVAYNARIGGVRMLDGEVTDAVEARSLGLNPNHIHIYSASWGPEDDGKTVDGPARLAEEAFFRGVSQGRGGLGSIFVWASGNGGREHDSCNCDGYTNSIYTLSISSATQFGNVPWYSEACSSTLATTYSSGNQNEKQIVTTDLRQKCTESHTGTSASAPLAAGIIALTLEANKNLTWRDMQHLVVQTSKPAHLNANDWATNGVGRKVSHSYGYGLLDAGAMVALAQNWTTVAPQRKCIIDILTEPKDIGKRLEVRKTVTACLGEPNHITRLEHAQARLTLSYNRRGDLAIHLVSPMGTRSTLLAARPHDYSADGFNDWAFTTTHSWDEDPSGEWVLEIENTSEANNYGTLTKFTLVLYGTAPEGLPVPPESSGCKTLTSSQACVVCEEGFSLHQKSCVQHCPPGFAPQVLDTHYSTENDVETIRASVCAPCHASCATCQGPALTDCLSCPSHASLDPVEQTCSRQSQSSRESPPQQQPPRLPPEVEAGQRLRAGLLPSHLPEVVAGLSCAFIVLVFVTVFLVLQLRSGFSFRGVKVYTMDRGLISYKGLPPEAWQEECPSDSEEDEGRGERTAFIKDQSAL

SEQ ID NO: 12 - DNA, 2405 bp, Artificial Sequence
  DNA sequence encoding Furin (ID 863)
gctagccaccatggagctgaggccctggttgctatgggtggtagcagcaacaggaaccttggtcctgctagcagctgatgctcagggccagaaggtcttcaccaacacgtgggctgtgcgcatccctggaggcccagcggtggccaacagtgtggcacggaagcatgggttcctcaacctgggccagatcttcggggactattaccacttctggcatcgaggagtgacgaagcggtccctgtcgcctcaccgcccgcggcacagccggctgcagagggagcctcaagtacagtggctggaacagcaggtggcaaagcgacggactaaacgggacgtgtaccaggagcccacagaccccaagtttcctcagcagtggtacctgtctggtgtcactcagcgggacctgaatgtgaaggcggcctgggcgcagggctacacagggcacggcattgtggtctccattctggacgatggcatcgagaagaaccacccggacttggcaggcaattatgatcctggggccagttttgatgtcaatgaccaggaccctgacccccagcctcggtacacacagatgaatgacaacaggcacggcacacggtgtgcgggggaagtggctgcggtggccaacaacggtgtctgtggtgtaggtgtggcctacaatgcccgcattggaggggtgcgcatgctggatggcgaggtgacagatgcagtggaggcacgctcgctgggcctgaaccccaaccacatccacatctacagtgccagctggggccccgaggatgacggcaagacagtggatgggccagcccgcctcgccgaggaggccttcttccgtggggttagccagggccgaggggggctgggctccatctttgtctgggcctcggggaacgggggccgggaacatgacagctgcaactgcgacggctacaccaacagtatctacacgctgtccatcagcagcgccacgcagtttggcaacgtgccgtggtacagcgaggcctgctcgtccacactggccacgacctacagcagtggcaaccagaatgagaagcagatcgtgacgactgacttgcggcagaagtgcacggagtctcacacgggcacctcagcctctgcccccttagcagccggcatcattgctctcaccctggaggccaataagaacctcacatggcgggacatgcaacacctggtggtacagacctcgaagccagcccacctcaatgccaacgactgggccaccaatggtgtgggccggaaagtgagccactcatatggctacgggcttttggacgcaggcgccatggtggccctggcccagaattggaccacagtggccccccagcggaagtgcatcatcgacatcctcaccgagcccaaagacatcgggaaacggctcgaggtgcggaagaccgtgaccgcgtgcctgggcgagcccaaccacatcactcggctggagcacgctcaggcgcggctcaccctgtcctataatcgccgtggcgacctggccatccacctggtcagccccatgggcacccgctccaccctgctggcagccaggccacatgactactccgcagatgggtttaatgactgggccttcacgacaactcattcctgggatgaggatccctctggcgagtgggtcctagagattgaaaacaccagcgaagccaacaactatgggacgctgaccaagttcaccctcgtactctatggcaccgcccctgaggggctgcccgtacctccagaaagcagtggctgcaagaccctcacgtccagtcaggcctgtgtggtgtgcgaggaaggcttctccctgcaccagaagagctgtgtccagcactgccccccaggcttcgccccccaagtcctcgatacgcactatagcaccgagaatgacgtggagaccatccgggccagcgtctgcgccccctgccacgcctcatgtgccacatgccaggggccggccctgacagactgcctcagctgccccagccacgcctccttggaccctgtggagcagacttgctcccggcaaagccagagcagccgagagtccccgccacagcagcagccacctcggctgcccccggaggtggaggcggggcaacggctgcgggcagggctgctgccctcacacctgcctgaggtggtggccggcctcagctgcgccttcatcgtgctggtcttcgtcactgtcttcctggtcctgcagctgcgctctggctttagttttcggggggtgaaggtgtacaccatggaccgtggcctcatctcctacaaggggctgccccctgaagcctggcaggaggagtgcccgtctgactcagaagaggacgagggccggggcgagaggaccgcctttatcaaagaccagagcgccctctgatagcggccgc

SEQ ID NO: 13 - protein, 1859 aa, Artificial Sequence
  Amino acid sequence of D1D2D`D3-HSA (CSL626)
MIPARFAGVLLALALILPGTLCAEGTRGRSSTARCSLFGSDFVNTFDGSMYSFAGYCSYLLAGGCQKRSFSIIGDFQNGKRVSLSVYLGEFFDIHLFVNGTVTQGDQRVSMPYASKGLYLETEAGYYKLSGEAYGFVARIDGSGNFQVLLSDRYFNKTCGLCGNFNIFAEDDFMTQEGTLTSDPYDFANSWALSSGEQWCERASPPSSSCNISSGEMQKGLWEQCQLLKSTSVFARCHPLVDPEPFVALCEKTLCECAGGLECACPALLEYARTCAQEGMVLYGWTDHSACSPVCPAGMEYRQCVSPCARTCQSLHINEMCQERCVDGCSCPEGQLLDEGLCVESTECPCVHSGKRYPPGTSLSRDCNTCICRNSQWICSNEECPGECLVTGQSHFKSFDNRYFTFSGICQYLLARDCQDHSFSIVIETVQCADDRDAVCTRSVTVRLPGLHNSLVKLKHGAGVAMDGQDVQLPLLKGDLRIQHTVTASVRLSYGEDLQMDWDGRGRLLVKLSPVYAGKTCGLCGNYNGNQGDDFLTPSGLAEPRVEDFGNAWKLHGDCQDLQKQHSDPCALNPRMTRFSEEACAVLTSPTFEACHRAVSPLPYLRNCRYDVCSCSDGRECLCGALASYAAACAGRGVRVAWREPGRCELNCPKGQVYLQCGTPCNLTCRSLSYPDEECNEACLEGCFCPPGLYMDERGDCVPKAQCPCYYDGEIFQPEDIFSDHHTMCYCEDGFMHCTMSGVPGSLLPDAVLSSPLSHRSKRSLSCRPPMVKLVCPADNLRAEGLECTKTCQNYDLECMSMGCVSGCLCPPGMVRHENRCVALERCPCFHQGKEYAPGETVKIGCNTCVCRDRKWNCTDHVCDATCSTIGMAHYLTFDGLKYLFPGECQYVLVQDYCGSNPGTFRILVGNKGCSHPSVKCKKRVTILVEGGEIELFDGEVNVKRPMKDETHFEVVESGRYIILLLGKALSVVWDRHLSISVVLKQTYQEKVCGLCGNFDGIQNNDLTSSNLQVEEDPVDFGNSWKVSSQCADTRKVPLDSSPATCHNNIMKQTMVDSSCRILTSDVFQDCNKLVDPEPYLDVCIYDTCSCESIGDCACFCDTIAAYAHVCAQHGKVVTWRTATLCPQSCEERNLRENGYECEWRYNSCAPACQVTCQHPEPLACPVQCVEGCHAHCPPGKILDELLQTCVDPEDCPVCEVAGRRFASGKKVTLNPSDPEHCQICHCDVVNLTCEACQEPGGSSGGSGGSGGSGGSGGSGGSGGSGGSGGSGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLT

SEQ ID NO: 14 - protein, 2136 aa, Artificial Sequence; identical to SEQ ID NO: 7
  Amino acid sequence of D1D2D`D3(EYA)-HSA (CSL629)

SEQ ID NO: 15 - protein, 1444 aa, Artificial Sequence
  Amino acid sequence of secreted rVIII- SingleChain (CSL627)
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 16 - protein, 247 aa, Artificial Sequence
  Amino acid sequence of anti-TER119 single chain fragment variable
EVKLQESGGGLVQPGGSLKLSCVASGFTFRDHWMNWVRQAPGKTMEWIGDIRPDGSDTNYAPSVRNRFTISRDNARSILYLQMSNMRSDYTATYYCVRDSPTRAGLMDAWGQGTSVTVSSGGGGSGGGGSGGGGSGGGGSDIQMTQSPSVLSASVGDRVTLNCKASQNINKYLNWYQQKLGEAPKVLIYNTNNLQTGIPSRFSGSGSGTDFTLTISSLQPEDFATYFCFQHYTWPTFGGGTKLEIKR

SEQ ID NO: 17 - DNA, 8442 bp, Homo sapiens
  CDS (1)..(8442)
atgattcctgccagatttgccggggtgctgcttgctctggccctcattttgccagggaccctttgtgcagaaggaactcgcggcaggtcatccacggcccgatgcagccttttcggaagtgacttcgtcaacacctttgatgggagcatgtacagctttgcgggatactgcagttacctcctggcagggggctgccagaaacgctccttctcgattattggggacttccagaatggcaagagagtgagcctctccgtgtatcttggggaattttttgacatccatttgtttgtcaatggtaccgtgacacagggggaccaaagagtctccatgccctatgcctccaaagggctgtatctagaaactgaggctgggtactacaagctgtccggtgaggcctatggctttgtggccaggatcgatggcagcggcaactttcaagtcctgctgtcagacagatacttcaacaagacctgcgggctgtgtggcaactttaacatctttgctgaagatgactttatgacccaagaagggaccttgacctcggacccttatgactttgccaactcatgggctctgagcagtggagaacagtggtgtgaacgggcatctcctcccagcagctcatgcaacatctcctctggggaaatgcagaagggcctgtgggagcagtgccagcttctgaagagcacctcggtgtttgcccgctgccaccctctggtggaccccgagccttttgtggccctgtgtgagaagactttgtgtgagtgtgctggggggctggagtgcgcctgccctgccctcctggagtacgcccggacctgtgcccaggagggaatggtgctgtacggctggaccgaccacagcgcgtgcagcccagtgtgccctgctggtatggagtataggcagtgtgtgtccccttgcgccaggacctgccagagcctgcacatcaatgaaatgtgtcaggagcgatgcgtggatggctgcagctgccctgagggacagctcctggatgaaggcctctgcgtggagagcaccgagtgtccctgcgtgcattccggaaagcgctaccctcccggcacctccctctctcgagactgcaacacctgcatttgccgaaacagccagtggatctgcagcaatgaagaatgtccaggggagtgccttgtcacaggtcaatcacacttcaagagctttgacaacagatacttcaccttcagtgggatctgccagtacctgctggcccgggattgccaggaccactccttctccattgtcattgagactgtccagtgtgctgatgaccgcgacgctgtgtgcacccgctccgtcaccgtccggctgcctggcctgcacaacagccttgtgaaactgaagcatggggcaggagttgccatggatggccaggacgtccagctccccctcctgaaaggtgacctccgcatccagcatacagtgacggcctccgtgcgcctcagctacggggaggacctgcagatggactgggatggccgcgggaggctgctggtgaagctgtcccccgtctatgccgggaagacctgcggcctgtgtgggaattacaatggcaaccagggcgacgacttccttaccccctctgggctggcggagccccgggtggaggacttcgggaacgcctggaagctgcacggggactgccaggacctgcagaagcagcacagcgatccctgcgccctcaacccgcgcatgaccaggttctccgaggaggcgtgcgcggtcctgacgtcccccacattcgaggcctgccatcgtgccgtcagcccgctgccctacctgcggaactgccgctacgacgtgtgctcctgctcggacggccgcgagtgcctgtgcggcgccctggccagctatgccgcggcctgcgcggggagaggcgtgcgcgtcgcgtggcgcgagccaggccgctgtgagctgaactgcccgaaaggccaggtgtacctgcagtgcgggaccccctgcaacctgacctgccgctctctctcttacccggatgaggaatgcaatgaggcctgcctggagggctgcttctgccccccagggctctacatggatgagaggggggactgcgtgcccaaggcccagtgcccctgttactatgacggtgagatcttccagccagaagacatcttctcagaccatcacaccatgtgctactgtgaggatggcttcatgcactgtaccatgagtggagtccccggaagcttgctgcctgacgctgtcctcagcagtcccctgtctcatcgcagcaaaaggagcctatcctgtcggccccccatggtcaagctggtgtgtcccgctgacaacctgcgggctgaagggctcgagtgtaccaaaacgtgccagaactatgacctggagtgcatgagcatgggctgtgtctctggctgcctctgccccccgggcatggtccggcatgagaacagatgtgtggccctggaaaggtgtccctgcttccatcagggcaaggagtatgcccctggagaaacagtgaagattggctgcaacacttgtgtctgtcgggaccggaagtggaactgcacagaccatgtgtgtgatgccacgtgctccacgatcggcatggcccactacctcaccttcgacgggctcaaatacctgttccccggggagtgccagtacgttctggtgcaggattactgcggcagtaaccctgggacctttcggatcctagtggggaataagggatgcagccacccctcagtgaaatgcaagaaacgggtcaccatcctggtggagggaggagagattgagctgtttgacggggaggtgaatgtgaagaggcccatgaaggatgagactcactttgaggtggtggagtctggccggtacatcattctgctgctgggcaaagccctctccgtggtctgggaccgccacctgagcatctccgtggtcctgaagcagacataccaggagaaagtgtgtggcctgtgtgggaattttgatggcatccagaacaatgacctcaccagcagcaacctccaagtggaggaagaccctgtggactttgggaactcctggaaagtgagctcgcagtgtgctgacaccagaaaagtgcctctggactcatcccctgccacctgccataacaacatcatgaagcagacgatggtggattcctcctgtagaatccttaccagtgacgtcttccaggactgcaacaagctggtggaccccgagccatatctggatgtctgcatttacgacacctgctcctgtgagtccattggggactgcgcctgcttctgcgacaccattgctgcctatgcccacgtgtgtgcccagcatggcaaggtggtgacctggaggacggccacattgtgcccccagagctgcgaggagaggaatctccgggagaacgggtatgagtgtgagtggcgctataacagctgtgcacctgcctgtcaagtcacgtgtcagcaccctgagccactggcctgccctgtgcagtgtgtggagggctgccatgcccactgccctccagggaaaatcctggatgagcttttgcagacctgcgttgaccctgaagactgtccagtgtgtgaggtggctggccggcgttttgcctcaggaaagaaagtcaccttgaatcccagtgaccctgagcactgccagatttgccactgtgatgttgtcaacctcacctgtgaagcctgccaggagccgggaggcctggtggtgcctcccacagatgccccggtgagccccaccactctgtatgtggaggacatctcggaaccgccgttgcacgatttctactgcagcaggctactggacctggtcttcctgctggatggctcctccaggctgtccgaggctgagtttgaagtgctgaaggcctttgtggtggacatgatggagcggctgcgcatctcccagaagtgggtccgcgtggccgtggtggagtaccacgacggctcccacgcctacatcgggctcaaggaccggaagcgaccgtcagagctgcggcgcattgccagccaggtgaagtatgcgggcagccaggtggcctccaccagcgaggtcttgaaatacacactgttccaaatcttcagcaagatcgaccgccctgaagcctcccgcatcgccctgctcctgatggccagccaggagccccaacggatgtcccggaactttgtccgctacgtccagggcctgaagaagaagaaggtcattgtgatcccggtgggcattgggccccatgccaacctcaagcagatccgcctcatcgagaagcaggcccctgagaacaaggccttcgtgctgagcagtgtggatgagctggagcagcaaagggacgagatcgttagctacctctgtgaccttgcccctgaagcccctcctcctactctgcccccccacatggcacaagtcactgtgggcccggggctcttgggggtttcgaccctggggcccaagaggaactccatggttctggatgtggcgttcgtcctggaaggatcggacaaaattggtgaagccgacttcaacaggagcaaggagttcatggaggaggtgattcagcggatggatgtgggccaggacagcatccacgtcacggtgctgcagtactcctacatggtgaccgtggagtaccccttcagcgaggcacagtccaaaggggacatcctgcagcgggtgcgagagatccgctaccagggcggcaacaggaccaacactgggctggccctgcggtacctctctgaccacagcttcttggtcagccagggtgaccgggagcaggcgcccaacctggtctacatggtcaccggaaatcctgcctctgatgagatcaagaggctgcctggagacatccaggtggtgcccattggagtgggccctaatgccaacgtgcaggagctggagaggattggctggcccaatgcccctatcctcatccaggactttgagacgctcccccgagaggctcctgacctggtgctgcagaggtgctgctccggagaggggctgcagatccccaccctctcccctgcacctgactgcagccagcccctggacgtgatccttctcctggatggctcctccagtttcccagcttcttattttgatgaaatgaagagtttcgccaaggctttcatttcaaaagccaatatagggcctcgtctcactcaggtgtcagtgctgcagtatggaagcatcaccaccattgacgtgccatggaacgtggtcccggagaaagcccatttgctgagccttgtggacgtcatgcagcgggagggaggccccagccaaatcggggatgccttgggctttgctgtgcgatacttgacttcagaaatgcatggggcgcgcccgggagcctcaaaggcggtggtcatcctggtcacggacgtctctgtggattcagtggatgcagcagctgatgccgccaggtccaacagagtgacagtgttccctattggaattggagatcgctacgatgcagcccagctacggatcttggcaggcccagcaggcgactccaacgtggtgaagctccagcgaatcgaagacctccctaccatggtcaccttgggcaattccttcctccacaaactgtgctctggatttgttaggatttgcatggatgaggatgggaatgagaagaggcccggggacgtctggaccttgccagaccagtgccacaccgtgacttgccagccagatggccagaccttgctgaagagtcatcgggtcaactgtgaccgggggctgaggccttcgtgccctaacagccagtcccctgttaaagtggaagagacctgtggctgccgctggacctgcccctgcgtgtgcacaggcagctccactcggcacatcgtgacctttgatgggcagaatttcaagctgactggcagctgttcttatgtcctatttcaaaacaaggagcaggacctggaggtgattctccataatggtgcctgcagccctggagcaaggcagggctgcatgaaatccatcgaggtgaagcacagtgccctctccgtcgagctgcacagtgacatggaggtgacggtgaatgggagactggtctctgttccttacgtgggtgggaacatggaagtcaacgtttatggtgccatcatgcatgaggtcagattcaatcaccttggtcacatcttcacattcactccacaaaacaatgagttccaactgcagctcagccccaagacttttgcttcaaagacgtatggtctgtgtgggatctgtgatgagaacggagccaatgacttcatgctgagggatggcacagtcaccacagactggaaaacacttgttcaggaatggactgtgcagcggccagggcagacgtgccagcccatcctggaggagcagtgtcttgtccccgacagctcccactgccaggtcctcctcttaccactgtttgctgaatgccacaaggtcctggctccagccacattctatgccatctgccagcaggacagttgccaccaggagcaagtgtgtgaggtgatcgcctcttatgcccacctctgtcggaccaacggggtctgcgttgactggaggacacctgatttctgtgctatgtcatgcccaccatctctggtttataaccactgtgagcatggctgtccccggcactgtgatggcaacgtgagctcctgtggggaccatccctccgaaggctgtttctgccctccagataaagtcatgttggaaggcagctgtgtccctgaagaggcctgcactcagtgcattggtgaggatggagtccagcaccagttcctggaagcctgggtcccggaccaccagccctgtcagatctgcacatgcctcagcgggcggaaggtcaactgcacaacgcagccctgccccacggccaaagctcccacgtgtggcctgtgtgaagtagcccgcctccgccagaatgcagaccagtgctgccccgagtatgagtgtgtgtgtgacccagtgagctgtgacctgcccccagtgcctcactgtgaacgtggcctccagcccacactgaccaaccctggcgagtgcagacccaacttcacctgcgcctgcaggaaggaggagtgcaaaagagtgtccccaccctcctgccccccgcaccgtttgcccacccttcggaagacccagtgctgtgatgagtatgagtgtgcctgcaactgtgtcaactccacagtgagctgtccccttgggtacttggcctcaaccgccaccaatgactgtggctgtaccacaaccacctgccttcccgacaaggtgtgtgtccaccgaagcaccatctaccctgtgggccagttctgggaggagggctgcgatgtgtgcacctgcaccgacatggaggatgccgtgatgggcctccgcgtggcccagtgctcccagaagccctgtgaggacagctgtcggtcgggcttcacttacgttctgcatgaaggcgagtgctgtggaaggtgcctgccatctgcctgtgaggtggtgactggctcaccgcggggggactcccagtcttcctggaagagtgtcggctcccagtgggcctccccggagaacccctgcctcatcaatgagtgtgtccgagtgaaggaggaggtctttatacaacaaaggaacgtctcctgcccccagctggaggtccctgtctgcccctcgggctttcagctgagctgtaagacctcagcgtgctgcccaagctgtcgctgtgagcgcatggaggcctgcatgctcaatggcactgtcattgggcccgggaagactgtgatgatcgatgtgtgcacgacctgccgctgcatggtgcaggtgggggtcatctctggattcaagctggagtgcaggaagaccacctgcaacccctgccccctgggttacaaggaagaaaataacacaggtgaatgttgtgggagatgtttgcctacggcttgcaccattcagctaagaggaggacagatcatgacactgaagcgtgatgagacgctccaggatggctgtgatactcacttctgcaaggtcaatgagagaggagagtacttctgggagaagagggtcacaggctgcccaccctttgatgaacacaagtgtctggctgagggaggtaaaattatgaaaattccaggcacctgctgtgacacatgtgaggagcctgagtgcaacgacatcactgccaggctgcagtatgtcaaggtgggaagctgtaagtctgaagtagaggtggatatccactactgccagggcaaatgtgccagcaaagccatgtactccattgacatcaacgatgtgcaggaccagtgctcctgctgctctccgacacggacggagcccatgcaggtggccctgcactgcaccaatggctctgttgtgtaccatgaggttctcaatgccatggagtgcaaatgctcccccaggaagtgcagcaagtga

SEQ ID NO: 18 - protein, 2813 aa, Homo sapiens
MIPARFAGVLLALALILPGTLCAEGTRGRSSTARCSLFGSDFVNTFDGSMYSFAGYCSYLLAGGCQKRSFSIIGDFQNGKRVSLSVYLGEFFDIHLFVNGTVTQGDQRVSMPYASKGLYLETEAGYYKLSGEAYGFVARIDGSGNFQVLLSDRYFNKTCGLCGNFNIFAEDDFMTQEGTLTSDPYDFANSWALSSGEQWCERASPPSSSCNISSGEMQKGLWEQCQLLKSTSVFARCHPLVDPEPFVALCEKTLCECAGGLECACPALLEYARTCAQEGMVLYGWTDHSACSPVCPAGMEYRQCVSPCARTCQSLHINEMCQERCVDGCSCPEGQLLDEGLCVESTECPCVHSGKRYPPGTSLSRDCNTCICRNSQWICSNEECPGECLVTGQSHFKSFDNRYFTFSGICQYLLARDCQDHSFSIVIETVQCADDRDAVCTRSVTVRLPGLHNSLVKLKHGAGVAMDGQDVQLPLLKGDLRIQHTVTASVRLSYGEDLQMDWDGRGRLLVKLSPVYAGKTCGLCGNYNGNQGDDFLTPSGLAEPRVEDFGNAWKLHGDCQDLQKQHSDPCALNPRMTRFSEEACAVLTSPTFEACHRAVSPLPYLRNCRYDVCSCSDGRECLCGALASYAAACAGRGVRVAWREPGRCELNCPKGQVYLQCGTPCNLTCRSLSYPDEECNEACLEGCFCPPGLYMDERGDCVPKAQCPCYYDGEIFQPEDIFSDHHTMCYCEDGFMHCTMSGVPGSLLPDAVLSSPLSHRSKRSLSCRPPMVKLVCPADNLRAEGLECTKTCQNYDLECMSMGCVSGCLCPPGMVRHENRCVALERCPCFHQGKEYAPGETVKIGCNTCVCRDRKWNCTDHVCDATCSTIGMAHYLTFDGLKYLFPGECQYVLVQDYCGSNPGTFRILVGNKGCSHPSVKCKKRVTILVEGGEIELFDGEVNVKRPMKDETHFEVVESGRYIILLLGKALSVVWDRHLSISVVLKQTYQEKVCGLCGNFDGIQNNDLTSSNLQVEEDPVDFGNSWKVSSQCADTRKVPLDSSPATCHNNIMKQTMVDSSCRILTSDVFQDCNKLVDPEPYLDVCIYDTCSCESIGDCACFCDTIAAYAHVCAQHGKVVTWRTATLCPQSCEERNLRENGYECEWRYNSCAPACQVTCQHPEPLACPVQCVEGCHAHCPPGKILDELLQTCVDPEDCPVCEVAGRRFASGKKVTLNPSDPEHCQICHCDVVNLTCEACQEPGGLVVPPTDAPVSPTTLYVEDISEPPLHDFYCSRLLDLVFLLDGSSRLSEAEFEVLKAFVVDMMERLRISQKWVRVAVVEYHDGSHAYIGLKDRKRPSELRRIASQVKYAGSQVASTSEVLKYTLFQIFSKIDRPEASRIALLLMASQEPQRMSRNFVRYVQGLKKKKVIVIPVGIGPHANLKQIRLIEKQAPENKAFVLSSVDELEQQRDEIVSYLCDLAPEAPPPTLPPHMAQVTVGPGLLGVSTLGPKRNSMVLDVAFVLEGSDKIGEADFNRSKEFMEEVIQRMDVGQDSIHVTVLQYSYMVTVEYPFSEAQSKGDILQRVREIRYQGGNRTNTGLALRYLSDHSFLVSQGDREQAPNLVYMVTGNPASDEIKRLPGDIQVVPIGVGPNANVQELERIGWPNAPILIQDFETLPREAPDLVLQRCCSGEGLQIPTLSPAPDCSQPLDVILLLDGSSSFPASYFDEMKSFAKAFISKANIGPRLTQVSVLQYGSITTIDVPWNVVPEKAHLLSLVDVMQREGGPSQIGDALGFAVRYLTSEMHGARPGASKAVVILVTDVSVDSVDAAADAARSNRVTVFPIGIGDRYDAAQLRILAGPAGDSNVVKLQRIEDLPTMVTLGNSFLHKLCSGFVRICMDEDGNEKRPGDVWTLPDQCHTVTCQPDGQTLLKSHRVNCDRGLRPSCPNSQSPVKVEETCGCRWTCPCVCTGSSTRHIVTFDGQNFKLTGSCSYVLFQNKEQDLEVILHNGACSPGARQGCMKSIEVKHSALSVELHSDMEVTVNGRLVSVPYVGGNMEVNVYGAIMHEVRFNHLGHIFTFTPQNNEFQLQLSPKTFASKTYGLCGICDENGANDFMLRDGTVTTDWKTLVQEWTVQRPGQTCQPILEEQCLVPDSSHCQVLLLPLFAECHKVLAPATFYAICQQDSCHQEQVCEVIASYAHLCRTNGVCVDWRTPDFCAMSCPPSLVYNHCEHGCPRHCDGNVSSCGDHPSEGCFCPPDKVMLEGSCVPEEACTQCIGEDGVQHQFLEAWVPDHQPCQICTCLSGRKVNCTTQPCPTAKAPTCGLCEVARLRQNADQCCPEYECVCDPVSCDLPPVPHCERGLQPTLTNPGECRPNFTCACRKEECKRVSPPSCPPHRLPTLRKTQCCDEYECACNCVNSTVSCPLGYLASTATNDCGCTTTTCLPDKVCVHRSTIYPVGQFWEEGCDVCTCTDMEDAVMGLRVAQCSQKPCEDSCRSGFTYVLHEGECCGRCLPSACEVVTGSPRGDSQSSWKSVGSQWASPENPCLINECVRVKEEVFIQQRNVSCPQLEVPVCPSGFQLSCKTSACCPSCRCERMEACMLNGTVIGPGKTVMIDVCTTCRCMVQVGVISGFKLECRKTTCNPCPLGYKEENNTGECCGRCLPTACTIQLRGGQIMTLKRDETLQDGCDTHFCKVNERGEYFWEKRVTGCPPFDEHKCLAEGGKIMKIPGTCCDTCEEPECNDITARLQYVKVGSCKSEVEVDIHYCQGKCASKAMYSIDINDVQDQCSCCSPTRTEPMQVALHCTNGSVVYHEVLNAMECKCSPRKCSK

SEQ ID NO: 19 - protein, 585 aa, Homo sapiens [sequence omitted]

SEQ ID NO: 20 - protein, 2332 aa, Homo sapiens
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHRSTRQKQFNATTIPENDIEKTDPWFAHRTPMPKIQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSSLGPPSMPVHYDSQLDTTLFGKKSSPLTESGGPLSLSEENNDSKLLESGLMNSQESSWGKNVSSTESGRLFKGKRAHGPALLTKDNALFKVSISLLKTNKTSNNSATNRKTHIDGPSLLIENSPSVWQNILESDTEFKKVTPLIHDRMLMDKNATALRLNHMSNKTTSSKNMEMVQQKKEGPIPPDAQNPDMSFFKMLFLPESARWIQRTHGKNSLNSGQGPSPKQLVSLGPEKSVEGQNFLSEKNKVVVGKGEFTKDVGLKEMVFPSSRNLFLTNLDNLHENNTHNQEKKIQEEIEKKETLIQENVVLPQIHTVTGTKNFMKNLFLLSTRQNVEGSYDGAYAPVLQDFRSLNDSTNRTKKHTAHFSKKGEEENLEGLGNQTKQIVEKYACTTRISPNTSQQNFVTQRSKRALKQFRLPLEETELEKRIIVDDTSTQWSKNMKHLTPSTLTQIDYNEKEKGAITQSPLSDCLTRSHSIPQANRSPLPIAKVSSFPSIRPIYLTRVLFQDNSSHLPAASYRKKDSGVQESSHFLQGAKKNNLSLAILTLEMTGDQREVGSLGTSATNSVTYKKVENTVLPKPDLPKTSGKVELLPKVHIYQKDLFPTETSNGSPGHLDLVEGSLLQGTEGAIKWNEANRPGKVPFLRVATESSAKTPSKLLDPLAWDNHYGTQIPKEEWKSQEKSPEKTAFKKKDTILSLNACESNHAIAAINEGQNKPEIEVTWAKQGRTERLCSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 21 - protein, 122 aa, Artificial sequence
  Amino acid sequence of a VHH nanobody designated IH4, disclosed in WO 2014/135528 A1
QVQLQESGGGSVQAGGSLRLSCVASGYTDSTYCVGWFRQAPGKEREGVARINTISGRPWYADSVKGRFTISQDNSKNTVFLQMNSLKPEDTAIYYCTLTTANSRGFCSGGYNYKGQGQVTVS

SEQ ID NO: 22 - DNA, 759 bp, Artificial Sequence
  DNA sequence encoding scFv 1C3, obtainable from hybridoma G26.4.1C3/86 [RAT 1C3/86] (ATCC? HB-9893)
gaagtcaggctcttggaatctggaggtggccctgtgcaaccaggcgggtccctgaagctttcttgtgcagcctccggattcgacttttcccggtactggatgaattgggtcaggagagctcccgggaaaggcctggaatggattggggagattaaccagcagtccagcaccatcaactacagtccacccctgaaggacaagttcatcatctctcgcgataatgccaaatcaaccctgtatctgcagatgaataaagtccgaagtgaggatactgctctctactactgtgcccggttgagccttactgctgctgggtttgcctattggggtcaggggacacttgtgaccgttagcgctggaggaggcgggtccggaggtggtggtagtggaggcggcgggagtggcggcggtggctctgacatcgtgatgtctcagagcccctcttcactggcagtgtccgttggggagaaggtgtctatgagctgcaagtcaagccagtctctgttcaacagtcgtaccaggaagaactatctcacttggtatcagcagaagcctggccaatcccctaaacccctgatctactgggcctcaacacgagaatccggcgtaccagaccgctttacgggctctggaagcggcacagacttcactctgaccataagctctgtgcaagcagaggatttggccgattattactgcaaacagtcctacaatctcagaacctttggcggcggcacaaagctggagattaagcggtaa

SEQ ID NO: 23 - protein, 252 aa, Artificial Sequence
  Amino acid sequence encoded by SEQ ID NO22
EVRLLESGGGPVQPGGSLKLSCAASGFDFSRYWMNWVRRAPGKGLEWIGEINQQSSTINYSPPLKDKFIISRDNAKSTLYLQMNKVRSEDTALYYCARLSLTAAGFAYWGQGTLVTVSAGGGGSGGGGSGGGGSGGGGSDIVMSQSPSSLAVSVGEKVSMSCKSSQSLFNSRTRKNYLTWYQQKPGQSPKPLIYWASTRESGVPDRFTGSGSGTDFTLTISSVQAEDLADYYCKQSYNLRTFGGGTKLEIKR

(19) United States
(12) Patent Application Publication
(10) Pub. No.: US 2022/0348637 A1
(43) Pub. Date: Nov. 3, 2022

(54) POLYPEPTIDES FOR INDUCING TOLERANCE TO FACTOR VIII

(71) Applicant: CSL BEHRING LENGNAU AG, Lengnau BE (CH)

(72) Inventors: Marco HOFMANN, Lahntal (DE); Adriana BAZ MORELLI, Carlton, Victoria (AU); Ingela VIKSTROM, Preston, Victoria (AU); Thomas WEIMER, Gladenbach (DE)

(21) Appl. No.: 17/775,704

(22) PCT Filed: Nov. 11, 2020
(86) PCT No.: PCT/EP2020/081698

§ 371 Date: May 10, 2022

(30) Foreign Application Priority Data
Nov. 11, 2019 (EP) 19208259.2

(51) Int. Cl. C07K 14/755; C12N 15/63; A61K 38/17

(52) U.S. Cl. CPC C07K 14/755; C12N 15/63; A61K 38/1741; C07K 2317/622

(57) ABSTRACT

The present invention relates to polypeptides comprising (i) a VWF moiety and (ii) an erythrocyte-binding moiety, wherein the polypeptide is capable of binding to blood coagulation factor VIII (FVIII). The erythrocyte-binding moiety may be selected from the group consisting of a peptide ligand, an antibody, an antibody fragment, and a single chain antigen binding domain (scFv). The polypeptides may be provided as a heterodimer. The polypeptides are useful for inducing tolerance to FVIII.

### FIELD OF THE INVENTION

The present invention relates to polypeptides that are useful for reducing the formation of antibodies to Factor VIII.

### BACKGROUND OF THE INVENTION

Haemophilia A is an inherited bleeding disorder characterized by plasma deficiency of coagulation factor VIII (FVIII). Hemophilia A patients are currently treated with FVIII replacement therapy. A major complication in 30% of patients is the occurrence of alloantibodies (inhibitors) that inactivate FVIII activity and may nullify replacement therapy. Patients with detectable inhibitors are treated with FVIII bypassing therapies (FIIe complex concentrates, rFVIIa, Emicizumab) and repeated doses of FVIII (ITI) and/or immune-suppressants. Such treatment is costly, requires repeated infusions and has a success rate of only 60%.

Immune tolerance is established in neonates and maintained during adult life through various physiological mechanisms to ensure lack of immune reactions against self-tissues. In Hemophilic boys, tolerance to FVIII is not sufficiently established because FVIII is not or incorrectly expressed through various mutations of the f8 gene locus—hence it is not recognized as a self-protein. In adult life, immune tolerance is induced and maintained by elimination or suppression of antigen-specific B and/or T cells. The main mechanisms involved in this process include antigen presentation by cells specialized in the induction of immune tolerance (liver or spleen macrophages), expansion of natural regulatory T cells (Tregs) or induction of antigen specific Tregs.

The inventors sought to formulate and administer FVIII in such a way that it can actively induce tolerance to FVIII in patients with inhibitors. Kontos S. et al. (2012; PNAS; 110(1):E60-E68) demonstrated that antigens targeted to apoptotic erythrocytes are cleared in the spleen and liver, and induce antigen-specific tolerogenic responses in CD4 and CD8 T cells. Glycophorin A is abundantly expressed on the surface of red blood cells (RBCs) and can be used to target Antigens (Ags) to the RBC surface. It turned out, however, that a fusion protein comprising FVIII and an erythrocyte-targeting moiety had only limited effect in reducing the formation of inhibitors.

There is a continued need for reducing inhibitor formation in the treatment of hemophilia A.

### SUMMARY OF THE INVENTION

The inventors of this application surprisingly found that a fusion protein comprising the D′D3 domain of von Willebrand Factor (VWF) and an erythrocyte-targeting moiety reduced inhibitor formation.

The present invention therefore relates to the subject matter defined in the following items [1] to [60]:
[1] A polypeptide comprising (i) a VWF moiety and (ii) an erythrocyte-binding moiety, wherein said polypeptide is capable of binding to blood coagulation factor VIII (FVIII).
[2] The polypeptide according to item [1], wherein said VWF moiety is capable of binding to FVIII.
[3] The polypeptide according to item [1], wherein said VWF moiety is a VWF or a fragment thereof, preferably human VWF or a fragment thereof.
[4] The polypeptide of any one of the preceding items, wherein said VWF moiety comprises the D′D3 domain of a VWF.
[5] The polypeptide of any one of the preceding items, wherein said VWF moiety comprises or substantially consists of a fragment of VWF.
[6] The polypeptide of any one of the preceding items, wherein said VWF moiety substantially consists of a truncated VWF.
[7] The polypeptide of any one of the preceding items, wherein the VWF moiety comprises an amino acid sequence having a sequence identity of at least 90% to amino acids 776 to 805 of SEQ ID NO:18; or an amino acid sequence having a sequence identity of at least 90% to amino acids 764 to 1242 of SEQ ID NO:18.
[8] The polypeptide of any one of the preceding items, wherein the VWF moiety lacks amino acids 1243 to 2813 of SEQ ID NO:18.
[9] The polypeptide of any one of the preceding items, wherein the VWF moiety consists of (a) amino acids 764 to 1242 of SEQ ID NO:18, of (b) an amino acid sequence having a sequence identity of at least 90% to amino acids 764 to 1242 of SEQ ID NO:18, or of (c) a fragment of (a) or (b).
[10] The polypeptide of any one of the preceding items, wherein said VWF moiety comprises at least one amino acid substitution as compared to the amino acid sequence of wild-type VWF as shown in SEQ ID NO:18.
[11] The polypeptide of item [10], wherein said at least one amino acid substitution increases the binding affinity to FVIII of the polypeptide, relative to a control polypeptide having the same sequence except for said at least one amino acid substitution.
[12] The polypeptide of item [10] or [11], wherein the at least one amino acid substitution is selected from the group of combinations consisting of S764G/S766Y, S764P/S766I, S764P/S766M, S764V/S766Y, S764E/S766Y, S764Y/S766Y, S764L/S766Y, S764P/S766W, S766W/S806A, S766Y/P769K, S766Y/P769N, S766Y/P769R, S764P/S766L, and S764E/S766Y/V1083A, referring to the sequence of SEQ ID NO:18 with regard to the amino acid numbering.
[13] The polypeptide of any one of items [10] to [12], wherein said at least one amino acid substitution is either the combination S764E/S766Y or S764E/S766Y/V1083A.
[14] The polypeptide of any one of the preceding items, wherein said polypeptide binds to said FVIII with a dissociation constant KD of 1 μM or less.
[15] The polypeptide of any one of the preceding items, wherein said polypeptide binds to said FVIII with a dissociation constant KD of 1 nM or less.
[16] The polypeptide of any one of the preceding items, wherein said polypeptide binds to said FVIII with a dissociation constant KD of 0.1 nM or less.
[17] The polypeptide of any one of the preceding items, wherein said polypeptide comprises a half-life extending moiety (HLEM).
[18] The polypeptide of item [17], wherein the HLEM is a heterologous amino acid sequence fused to the VWF moiety.
[19] The polypeptide of item [18], wherein said heterologous amino acid sequence comprises or consists of a protein or peptide selected from the group consisting of transferrin and fragments thereof, the C-terminal peptide of human chorionic gonadotropin, an XTEN sequence, homo-amino acid repeats (HAP), proline-alanine-serine repeats (PAS), albumin, afamin, alpha-fetoprotein, Vitamin D binding protein, polypeptides capable of binding under physiological conditions to albumin or immunoglobulin constant regions, polypeptides capable of binding to the neonatal Fc receptor (FcRn), particularly immunoglobulin constant regions and portions thereof, preferably the Fc portion of immunoglobulin, and combinations thereof.
[20] The polypeptide of item [17], wherein the HLEM is conjugated to the polypeptide comprising the VWF moiety.
[21] The polypeptide of item [20], wherein the HLEM is conjugated to the C-terminus of the polypeptide comprising the VWF moiety.
[22] The polypeptide of item [20] or [21], wherein said HLEM is selected from the group consisting of hydroxyethyl starch (HES), polyethylene glycol (PEG), polysialic acids (PSAs), elastin-like polypeptides, heparosan polymers, hyaluronic acid and non-proteinaceous albumin binding ligands, e.g. fatty acid chains, and combinations thereof.
[23] The polypeptide of item [17], wherein the HLEM is non-covalently linked to the polypeptide comprising the VWF moiety.
[24] The polypeptide of any one of items [1] to [20], wherein the polypeptide does not comprise any HLEM conjugated to the polypeptide.
[25] The polypeptide of any one of the preceding items, wherein said polypeptide is a glycoprotein comprising N-glycans, and wherein preferably at least 75%, preferably at least 85% of said N-glycans comprise, on average, at least one sialic acid moiety.
[26] The polypeptide of any one of the preceding items, wherein said polypeptide is present as a dimer or at least has a high proportion of dimers.
[27] The polypeptide of item [26], wherein at least 50%, or at least 70%, or at least 80%, or at least 90%, or at least 95% of said polypeptide is present as a dimer.
[28] The polypeptide of item [26] or [27], wherein the two monomers forming the dimer are covalently linked to each other via at least one or more disulfide bridges formed by cysteine residues within the VWF moiety.
[29] The polypeptide of item [28], wherein the cysteine residues forming the one or more disulfide bridges is/are selected from the group consisting of Cys-1099, Cys-1142, Cys-1222, Cys-1225, Cys-1227 and combinations thereof, preferably Cys-1099 and Cys-1142, wherein the amino acid numbering refers to SEQ ID NO:18.
[30] The polypeptide of any one of items [26] to [29], wherein the affinity of said dimer to FVIII is greater than the affinity of a monomeric polypeptide to FVIII, said monomeric polypeptide having the same amino acid sequence as a monomeric subunit of the dimeric polypeptide.
[31] The polypeptide of any one of items [26] to [30], wherein the ratio dimer:monomer of the polypeptide is at least 1.5, preferably at least 2, more preferably at least 2.5 or at least 3, or at least 4, or at least 5, or at least 10, or at least 20; or wherein the polypeptide does not comprise monomer and/or multimer forms of the polypeptide; or wherein the polypeptide is essentially free of monomer and/or multimer forms of the polypeptide.
[32] The polypeptide of any one of items [26] to [31], wherein the dimeric polypeptide has a FVIII binding affinity characterized by a dissociation constant K_{D} of less than 1 μM, preferably less than 1 nM, more preferably less than 500 pM, less than 200 pM, less than 100 pM, less than 90 pM or less than 80 pM.
[33] The polypeptide of item [32], wherein the KD ranges from 0.1 pM to 500 pM, from 0.5 pM to 200 pM, from 0.75 pM to 100 pM or most preferred from 1 pM to 80 pM.
[34] The polypeptide according to any one of items [26] to [33], wherein the polypeptide is a heterodimer.
[35] The polypeptide of item [34], wherein the heterodimer comprises a first subunit and a second subunit, wherein the first subunit comprises a first VWF moiety as defined in any one of the preceding items and the erythrocyte-binding moiety, and the second subunit comprises a second VWF moiety as defined in any one of the preceding items.
[36] The polypeptide of item [35], wherein said first VWF moiety and said second VWF moiety are identical.
[37] The polypeptide of item [35] or [36], wherein said second subunit does not comprise an erythrocyte-binding moiety.
[38] The polypeptide of any one of items [35] to [37], wherein said second subunit substantially consists of the second VWF moiety.
[39] The polypeptide of any one of the preceding items, wherein said erythrocyte-binding moiety is capable of binding to human erythrocytes.
[40], The polypeptide of any one of the preceding items, wherein said erythrocyte-binding moiety is capable of binding to a membrane protein on an erythrocyte, preferably to a human membrane protein on a human erythrocyte.
[41] The polypeptide of any one of the preceding items, wherein said erythrocyte-binding moiety is selected from the group consisting of a peptide ligand, an antibody, an antibody fragment, and a single chain antigen binding domain (scFv).
[42] The polypeptide of any one of the preceding items, wherein said erythrocyte-binding moiety is capable of specifically binding to a biomolecule selected from the group consisting of Band 3 (CD233), aquaporin-1, Glut-1, Kidd antigen, RhAg/Rh50 (CD241), Rh (CD240), Rh30CE (CD240CE), Rh30D (CD240D), Kx, glycophorin A (CD235a), glycophorin B (CD235b), glycophorin C (CD235c), glycophorin D (CD235d), Keil (CD238), Duffy/DARCi (CD234), CR1 (CD35), DAF (CD55), Globoside, CD44, ICAM-4 (CD242), Lu/B-CAM (CD239), XG1/XG2 (CD99), EMMPRIN/neurothelin (CD147), JMH, Glycosyltransferase, Cartwright, Dombrock, C4A/CAB, Scimma, MER2, stomatin, BA-I (CD24), GPIV (CD36), CD108, CD139, and Hantigen (CD173).
[43] A pharmaceutical composition comprising the polypeptide of any one of the preceding items and optionally a pharmaceutically acceptable carrier, diluent or excipient.
[44] The pharmaceutical composition of item [43], wherein the ratio dimer:monomer of the polypeptide in the composition is at least 1.5, preferably at least 2, more preferably at least 2.5 or at least 3, or at least 4, or at least 5, or at least 10, or at least 20; or wherein the composition does not comprise monomer and/or multimer forms of the polypeptide; or wherein the composition is essentially free of monomer and/or multimer forms of the polypeptide.
[45] The polypeptide of any one of items [1] to [42], or the pharmaceutical composition of item [43] or [44] for use in therapy.
[46] The polypeptide of any one of items [1] to [42], or the pharmaceutical composition of item [43] or [44] for use as a medicament.
[47] The polypeptide of any one of items [1] to [42], or the pharmaceutical composition of item [43] or [44] for use in the treatment of a blood coagulation disorder.
[48] The polypeptide for use according to item [47], or the pharmaceutical composition for use according to item [47], wherein said blood coagulation disorder is hemophilia A.
[49] The polypeptide for use according to item [47] or [48], or the pharmaceutical composition for use according to item [47] or [48], wherein said treatment comprises administering a FVIII to a subject.
[50] The polypeptide for use according to item [49], or the pharmaceutical composition for use according to item [49], wherein the polypeptide and the FVIII are co-administered.
[51] The polypeptide for use according to item [50], or the pharmaceutical composition for use according to item [50], wherein said co-administration is achieved either (i) by administration together in a single composition comprising the polypeptide and the FVIII, or (ii) by administration of the polypeptide and the FVIII each provided in separate compositions, wherein the polypeptide is administered before, after or concurrently with the FVIII.
[52] The polypeptide for use according to any one of items [49] to [51], or the pharmaceutical composition for use according to any one of items [49] to [51], wherein the ratio of polypeptide to FVIII is at least 1, or at least 2, or at least 4, or at least 10, or at least 20, or at least 50, or at least 100.
[53] The polypeptide of any one of items [1] to [42], or the pharmaceutical composition of item [43] or [44] for use in preventing or reducing inhibitor formation.
[54] The polypeptide for use according to item [53], or the pharmaceutical composition for use according to item [53], wherein said preventing or reducing inhibitor formation comprises administering (i) said polypeptide or said pharmaceutical composition and (ii) a FVIII to a subject.
[55] A nucleic acid encoding the polypeptide of any one of items [1] to [42].
[56] A plasmid or vector comprising the nucleic acid of item [55].
[57] A host cell comprising the plasmid or vector of item [56].
[58] A method of producing a polypeptide comprising a VWF and an erythrocyte-binding moiety, comprising (i) culturing the host cells of item [57] under conditions such that the polypeptide comprising the VWF and the erythrocyte-binding moiety are expressed; and (ii) optionally recovering the polypeptide comprising the VWF and the erythrocyte-binding moiety from the host cells or from the culture medium.
[59] A method of inducing tolerance to FVIII, comprising administering to a subject in need thereof, an effective amount of the polypeptide of any one of items [1] to [42].
[60] The polypeptide of any one of items [1] to [42], or the pharmaceutical composition of item [43] or [44] for use in the induction of tolerance to FVIII.

### DESCRIPTION OF THE FIGURES

FIG. 1: Scheme of D′D3-TER119scFv dimer. VWF-D′D3 FVIII binding domain was fused C-terminally with human albumin (HSA) and TER119scFv. D′D3 is expressed in line with D1D2, the VWF pro-peptide (not drawn). D1D2 is cleaved through co-expression of PACE/furin in same expression cell line. Dimers form through two interchain disulfides at C1099 and C1142 (exemplified through dotted lines).

FIG. 2: Dimer and monomer formation during expression. Western Blot of (F) mix of purified D′D3-FP dimer and monomer derived from CSL626, (T) D′D3-TER119 homodimer in supernatant of CHO culture, (M) marker. Tris Glycin 8-16%. Detection: anti-human serum albumin antibody (AP labeled).

FIG. 3: Binding of D′D3-TER119scFv to murine RBCs in vitro. Flow cytometry. Murine RBCs (1:100 whole blood) were stained by anti-TER119-PE MAb to define gate for single cell analysis (SSC/FSC). Then, murine RBCs were incubated with (A) PBS+1% BSA, (B) D′D3-TER119 dimer, 50 μg/ml, (C) D′D3-TER119 monomer, 50 μg/ml, (D) D′D3-TER119 monomer, 50 μg/ml, and anti-human albumin MAb, 20 μg/ml, (E) PBS+1% BSA and (F) anti-ter119-PE mAb, 0.2 μg/ml.

FIG. 4: Scheme of bispecific D′D3-TER119 heterodimer. A first VWF-D′D3 FVIII binding domain is fused C-terminally with human albumin (HSA) and TER119scFv. A second D′D3 is tagged at the C-terminus. D′D3 is expressed in line with D1D2, the VWF pro-peptide (not drawn). D1D2 is cleaved through co-expression of PACE/furin in same expression cell line. Dimers form through two inter-chain disulfide bonds at C1099 and C1142 (exemplified through dotted lines).

FIG. 5: Expression of D′133-TER119scFv heterodimers. Hetero-dimer were generated through co-expression of (A) D′D3-TER119scFv and D′D3 in a stably transfected cell line (SEQ ID NO:3 and SEQ ID NO:5). (B) Same expression strategy was applied using a high affinity variant of D′D3, D′D3(EYA), for both subunits (SEQ ID NO:7 and SEQ ID NO:9). SDS-PAGE (Commassie, Tris glycine) of protein upon CaptureSelect™ Human Albumin, Ni Sepharose and Superdex-200 steps. M: SeeBlue marker, TH: D′D3-TER119 heterodimer. F: Control D′D3-FP, monomer and dimer.

FIG. 6: rVIII-SingleChain binding to murine RBCs through D′D3-TER119 heterodimer in vitro. (A) D′D3-TER119 constructs were titrated and incubated with 12.5 IU/ml human rVIII-SingleChain. Human VWF (12.5 IU/ml) was added where indicated. Washed murine RBCs (1:100 whole blood) were added to protein mix at 37° C. Flow cytometry. RBCs gated by SSC/FSC and TER119-PE MAb. Detection: Polyclonal mouse anti-human FVIII-FITC, 20 μg/ml. D′D3_{WT}-TER119: n=1; D′D3_{EYA}-TER119: n=3 (independent experiments, two batches). (B) Exemplified fit of a Michaelis-Menten kinetic to calculate KM of D′D3_{EYA}-TER119±VWF using batch #2.

FIG. 7: Scheme of in vivo study plan. Prophylactic treatment of FVIII ko mice. FVIII KO mice (n=10), weekly, five intravenous injections. Treatment groups: rVIII-SingleChain (2000 IU/kg; 160 μg/kg) co-administered with D′D3_{EYA}-TER119 heterodimer (840 μg/kg, batch #1 or batch #2).

FIG. 8: Prophylactic treatment of FVIII ko mice. Results of anti-FVIII-antibody generation and its inhibitory effect in FVIII ko mice after administration of rVIII-SingleChain with or without D′D3_{EYA}-TER119. Control FVIII ko mice were treated with rVIII-SingleChain only. Test groups were treated with rVIII-SingleChain co-administered with D′D3_{EYA}-TER119. (A) Anti-FVIII-antibody generation (sum of extinction of ELISA OD) and (B) its inhibitory effect (Bethesda Units) in FVIII ko mice after administration of rVIII-SingleChain with or without D′D3_{EYA}-TER119 (batch 1 or batch 2). (C) Comparison of anti-FVIII-antibody generation and its inhibitory effect in FVIII ko mice after administration of rVIII-SingleChain with or without D′D3_{EYA}-TER119.

FIG. 9: Schematic representation of the treatment strategy to investigate long-term tolerance in FVIII ko mice. Treatment: (a) 1700 IU/kg rVIII-SingleChain and (b) co-administered with D′D3_{EYA}-TER119 heterodimer, 672 μg/kg. Four injections, i.v. weekly. Interim bleed at day 28. Re-challenge with 120 IU/kg at day 49 and 56. Terminal bleed and planned study end at day 63.

FIG. 10: Graphical representation showing the effect of D′D3_{EYA}-TER119 treatment in combination with rFVIII. Control (rVIII-SingleChain alone) and treatment group (rVIII-SingleChain+D′D3-TER119) at interim (day 28) and terminal (day 63) bleed: 1700 IU/kg rVIII-SingleChain alone and co-administered with D′D3_{EYA}-TER119 heterodimer, 672 μg/kg. Anti-FVIII antibodies were measured through (A) FVIII ADA ELISA. Interpretation was performed based on the predicted ratio OD₂₀₀(sample) over OD₂₀₀(standard) corresponding to the OD at dilution of 1/200 which was fitted for each titration. Statistical analysis used Kruskal-Wallis test with Dunn's multiple comparisons test; *p<0.05, *** p=0.0005, ns=not significant.

FIG. 11: Plasma (A) and pellet (B) FVIII levels in FVIII ko mice. FVIII chromogenic activity measured in plasma or centrifuged, re-suspended pellets from whole blood. Three groups of mice were treated with 200 IU/kg rVIII-SingleChain alone or co-administered with D′D3_{EYA}-FP (100 μg/kg, CSL629) or D′D3_{EYA}-TER119 heterodimer (84 μg/kg) at equal molar ratios of FVIII to its binding partner D′D3, 1 to 4. Samples were drawn 5 minutes, 3, 8, 16, 24, 48, 72 and 96 hours post administration. N=3. Data are given as mean and as modeled curves.

### DETAILED DESCRIPTION

The present invention relates to a polypeptide comprising (i) a VWF moiety and (ii) an erythrocyte-binding moiety, wherein said polypeptide is capable of binding to blood coagulation factor VIII (FVIII).

The VWF Moiety

The term “von Willebrand Factor” (VWF) as used herein includes naturally occurring (native) VWF, but also variants thereof, e.g. sequence variants where one or more residues have been inserted, deleted or substituted.

In one embodiment the VWF is human VWF represented by the amino acid sequence shown in SEQ ID NO:18. The cDNA encoding SEQ ID NO:18 is shown in SEQ ID NO:17.

The gene encoding human native VWF is transcribed into a 9 kb mRNA which is translated into a pre-propolypeptide of 2813 amino acids with an estimated molecular weight of 310,000 Da. The pre-propolypeptide contains an N-terminal 22 amino acids signal peptide, followed by a 741 amino acid pro-polypeptide (amino acids 23-763 of SEQ ID NO:18) and the mature subunit (amino acids 764-2813 of SEQ ID NO:18). Cleavage of the 741 amino acids propolypeptide from the N-terminus results in mature VWF consisting of 2050 amino acids. The amino acid sequence of the human native VWF pre-propolypeptide is shown in SEQ ID NO:18. Unless indicated otherwise, the amino acid numbering of VWF residues in this application refers to SEQ ID NO:18, even if the VWF molecule, in particular a truncated VWF, does not comprise all residues of SEQ ID NO:18.

The propolypeptide of native VWF comprises multiple domains. Different domain annotations can be found in the literature (see, e.g. Zhou et al. (2012) Blood 120(2): 449-458). The following domain annotation of native pre-propolypeptide of VWF is applied in this application: D1-D2-D′-D3-A1-A2-A3-D4-C1-C2-C3-C4-C5-C6-CK

With reference to SEQ ID NO:18, the D′ domain consists of amino acids 764-865; and the D3 domain consists of amino acids 866-1242.

The term “VWF moiety” as used herein refers to a peptide or polypeptide having amino acid sequence similarity to human VWF as shown in SEQ ID NO:18, or to a fragment thereof. The sequence similarity is preferably such that the sequence identity is at least 50%, or at least 60%, or at least 70%, or at least 80%, or at least 90%, or at least 95%, or at least 99%.

In one embodiment, the VWF moiety comprises or substantially consists of a truncated VWF. The feature “truncated” in terms of the present invention means that the polypeptide does not comprise the entire amino acid sequence of mature VWF (e.g. amino acids 764-2813 of SEQ ID NO:18). According to this embodiment, the VWF moiety does not comprise all amino acids 764-2813 of SEQ ID NO:18 but typically only a fragment thereof. A truncated VWF may also be referred to as a VWF fragment, or in the plural as VWF fragments.

Typically, the VWF moiety is capable of binding to a Factor VIII. Preferably, the VWF moiety is capable of binding to the mature form of human native Factor VIII. In another embodiment, the VWF moiety is capable of binding to a recombinant FVIII, e.g. to a B-domain deleted or single-chain FVIII. Binding of the VWF moiety to FVIII can be determined by a binding assay as described in Example 2 of WO 2010/087271 A1.

The polypeptide of the invention is capable of binding to FVIII. Preferably, the polypeptide of the invention is capable of binding to the mature form of human native Factor VIII. In another embodiment, the polypeptide of the invention is capable of binding to a recombinant FVIII, such as a B-domain deleted or single-chain FVIII. Binding of the polypeptide of the invention to FVIII can be determined by a binding assay as described in Example 2 of WO 2010/087271 A1.

The VWF moiety of the present invention preferably comprises or consists of an amino acid sequence having a sequence identity of at least 90% to amino acids 776 to 805 of SEQ ID NO:18 and is capable of binding to FVIII. In preferred embodiments the VWF moiety comprises or consists of an amino acid sequence having a sequence identity of at least 95%, at least 96%, at least 97%, at least 98%, or at least 99%, to amino acids 776 to 805 of SEQ ID NO:18 and is capable of binding to FVIII. In one embodiment, the VWF moiety comprises or consists of amino acids 776 to 805 of SEQ ID NO:18. Unless indicated otherwise herein, sequence identities are determined over the entire length of the reference sequence (e.g. amino acids 776 to 805 of SEQ ID NO:18).

The VWF moiety of the present invention preferably comprises or consists of an amino acid sequence having a sequence identity of at least 90% to amino acids 766 to 864 of SEQ ID NO:18 and is capable of binding to FVIII. In preferred embodiments the VWF moiety comprises or consists of an amino acid sequence having a sequence identity of at least 95%, at least 96%, at least 97%, at least 98%, or at least 99%, to amino acids 766 to 864 of SEQ ID NO:18 and is capable of binding to FVIII. In one embodiment, the VWF moiety comprises or consists of amino acids 766 to 864 of SEQ ID NO:18.

In another preferred embodiment, the VWF moiety consists of (a) an amino acid sequence having a sequence identity of at least 90% to amino acids 764 to 1242 of SEQ ID NO:18, or (b) a fragment thereof, provided that the VWF moiety is still capable of binding to FVIII. More preferably, the VWF moiety consists of (a) an amino acid sequence having a sequence identity of at least 95%, at least 96%, at least 97%, at least 98%, or at least 99%, to amino acids 764 to 1242 of SEQ ID NO:18, or (b) a fragment thereof, provided that the VWF moiety is still capable of binding to FVIII. In one embodiment, the VWF moiety consists of (a) amino acids 764 to 1242 of SEQ ID NO:18, or (b) a fragment thereof, provided that the VWF moiety is still capable of binding to FVIII.

As described in more detail below, the polypeptide of the invention may be prepared by a method which uses cells comprising a nucleic acid encoding the polypeptide comprising the VWF moiety. The nucleic acid is introduced into suitable host cells by techniques that are known per se.

In a preferred embodiment, the nucleic acid in the host cell encodes (a) an amino acid sequence having a sequence identity of at least 90% to amino acids 1 to 1242 of SEQ ID NO:18, or (b) a fragment thereof, provided that the VWF moiety is still capable of binding to FVIII. More preferably, the nucleic acid encodes (a) an amino acid sequence having a sequence identity of at least 95%, at least 96%, at least 97%, at least 98%, or at least 99%, to amino acids 1 to 1242 of SEQ ID NO:18, or (b) a fragment thereof, provided that the VWF moiety is still capable of binding to FVIII. In one embodiment, the nucleic acid encodes (a) amino acids 1 to 1242 of SEQ ID NO:18, or (b) a fragment thereof, provided that the VWF moiety is still capable of binding to FVIII. Especially if the polypeptide in accordance with this invention is a dimer, the nucleic acid will comprise a sequence also encoding amino acids 1 to 763 of VWF (e.g. SEQ ID NO:18), even if the VWF moiety in the polypeptide does not comprise amino acids 1 to 763 of VWF (e.g. SEQ ID NO:18).

The VWF moiety of the polypeptide of the invention according to a preferred embodiment may not comprise amino acid sequence 1 to 763 of VWF of SEQ ID NO:18.

According to further preferred embodiments, the VWF moiety comprises or consists of one of the following amino acid sequences, each referring to SEQ ID NO:18: 776-805; 766-805; 764-805; 776-810; 766-810; 764-810; 776-815; 766-815; 764-815; 776-820; 766-820; 764-820; 776-825; 766-825; 764-825; 776-830; 766-830; 764-830; 776-835; 766-835; 764-835; 776-840; 766-840; 764-840; 776-845; 766-845; 764-845; 776-850; 766-850; 764-850; 776-855; 766-855; 764-855; 776-860; 766-860; 764-860; 776-864; 766-864; 764-864; 776-865; 766-865; 764-865; 776-870; 766-870; 764-870; 776-875; 766-875; 764-875; 776-880; 766-880; 764-880; 776-885; 766-885; 764-885; 776-890; 766-890; 764-890; 776-895; 766-895; 764-895; 776-900; 766-900; 764-900; 776-905; 766-905; 764-905; 776-910; 766-910; 764-910; 776-915; 766-915; 764-915; 776-920; 766-920; 764-920; 776-925; 766-925; 764-925; 776-930; 766-930; 764-930; 776-935; 766-935; 764-935; 776-940; 766-940; 764-940; 776-945; 766-945; 764-945; 776-950; 766-950; 764-950; 776-955; 766-955; 764-955; 776-960; 766-960; 764-960; 776-965; 766-965; 764-965; 776-970; 766-970; 764-970; 776-975; 766-975; 764-975; 776-980; 766-980; 764-980; 776-985; 766-985; 764-985; 776-990; 766-990; 764-990; 776-995; 766-995; 764-995; 776-1000; 766-1000; 764-1000; 776-1005; 766-1005; 764-1005; 776-1010; 766-1010; 764-1010; 776-1015; 766-1015; 764-1015; 776-1020; 766-1020; 764-1020; 776-1025; 766-1025; 764-1025; 776-1030; 766-1030; 764-1030; 776-1035; 766-1035; 764-1035; 776-1040; 766-1040; 764-1040; 776-1045; 766-1045; 764-1045; 776-1050; 766-1050; 764-1050; 776-1055; 766-1055; 764-1055; 776-1060; 766-1060; 764-1060; 776-1065; 766-1065; 764-1065; 776-1070; 766-1070; 764-1070; 776-1075; 766-1075; 764-1075; 776-1080; 766-1080; 764-1080; 776-1085; 766-1085; 764-1085; 776-1090; 766-1090; 764-1090; 776-1095; 766-1095; 764-1095; 776-1100; 766-1100; 764-1100; 776-1105; 766-1105; 764-1105; 776-1110; 766-1110; 764-1110; 776-1115; 766-1115; 764-1115; 776-1120; 766-1120; 764-1120; 776-1125; 766-1125; 764-1125; 776-1130; 766-1130; 764-1130; 776-1135; 766-1135; 764-1135; 776-1140; 766-1140; 764-1140; 776-1145; 766-1145; 764-1145; 776-1150; 766-1150; 764-1150; 776-1155; 766-1155; 764-1155; 776-1160; 766-1160; 764-1160; 776-1165; 766-1165; 764-1165; 776-1170; 766-1170; 764-1170; 776-1175; 766-1175; 764-1175; 776-1180; 766-1180; 764-1180; 776-1185; 766-1185; 764-1185; 776-1190; 766-1190; 764-1190; 776-1195; 766-1195; 764-1195; 776-1200; 766-1200; 764-1200; 776-1205; 766-1205; 764-1205; 776-1210; 766-1210; 764-1210; 776-1215; 766-1215; 764-1215; 776-1220; 766-1220; 764-1220; 776-1225; 766-1225; 764-1225; 776-1230; 766-1230; 764-1230; 776-1235; 766-1235; 764-1235; 776-1240; 766-1240; 764-1240; 776-1242; 766-1242; 764-1242; 764-1464; 764-1250; 764-1041; 764-828; 764-865; 764-1045; 764-1035; 764-1128; 764-1198; 764-1268; 764-1261; 764-1264; 764-1459; 764-1463; 764-1464; 764-1683; 764-1873; 764-1482; 764-1479; 764-1672; and 764-1874.

In other embodiments the VWF moiety comprises or consists of an amino acid sequence that has a sequence identity of at least 90%, or at least 91%, or at least 92%, or at least 93%, or at least 94%, or at least 95%, or at least 96%, or at least 97%, or at least 98%, or at least 99%, to one of the amino acid sequences recited in the preceding paragraph, provided that the VWF moiety is capable of binding to FVIII.

In certain embodiments the VWF moiety has an internal deletion relative to mature wild type VWF. For example, the A1, A2, A3, D4, C1, C2, C3, C4, C5, C6, CK domains or combinations thereof may be deleted, and the D′ domain and/or the D3 domain is retained. According to further embodiments, the VWF moiety lacks one or more of the domains A1, A2, A3, D4, C1, C2, C3, C4, C5, C6 or CK. According to further embodiments, the VWF moiety lacks amino acids 1243 to 2813 of SEQ ID NO:4, i.e. the domains A1-A2-A3-D4-C1-C2-C3-C4-C5-C6-CK.

In further embodiments the VWF moiety or the polypeptide of the invention does not comprise the binding sites for platelet glycoprotein Ibα (GPIbα), collagen and/or integrin αIIbβIII (RGDS sequence within the C1 domain). In other embodiments, the VWF moiety or the polypeptide of the invention does not comprise the cleavage site (Tyr1605-Met1606) for ADAMTS13 which is located at the central A2 domain of VWF. In yet another embodiment, the VWF moiety or the polypeptide of the invention does not comprise the binding sites for GPIbα, and/or does not comprise the binding site(s) for collagen, and/or does not comprise the binding site for integrin αIIbβIII, and/or it does not comprise the cleavage site (Tyr1605-Met1606) for ADAMTS13 which is located at the central A2 domain of VWF. In a preferred embodiment the VWF moiety or the polypeptide of the invention does not comprise amino acids 1691 to 1905 of SEQ ID NO:18. In another preferred embodiment the VWF moiety or the polypeptide of the invention does not comprise amino acids 1691 to 1905 of the amino acid sequence deposited as UniProtKB-P04275. In another preferred embodiment the VWF moiety or the polypeptide of the invention does not comprise amino acids 1691 to 1905 of human VWF.

In another embodiment the polypeptide does not contain the VWF domains A1 and A3 or a part thereof and does have low or essentially no affinity for collagen type I and type III, said low or essentially no affinity being characterized by a dissociation constant K_{D}>10 μM for binding of the polypeptide to collagen type I and type III.

A polypeptide of the invention is termed a “dimer” in the present invention if two monomers of the polypeptide of the invention are linked covalently. Preferably, the covalent bond is located within the VWF moiety of the polypeptide of the invention. Preferably, the two monomeric subunits are covalently linked via at least one disulfide bridge, e.g. by one, two, three or four disulfide bridges. The cysteine residues forming the at least one disulfide bridge are preferably located within the VWF moiety of the polypeptide of the invention. In one embodiment, these cysteine residues are Cys-1099, Cys-1142, Cys-1222, Cys-1225, or Cys-1227 or combinations thereof. Preferably, the dimeric polypeptide of the invention does not comprise any further covalent bond linking the monomers in addition to said covalent bond located within the VWF moiety of the polypeptide, in particular does not comprise any further covalent bond located within the HLEM or HLEP portion of the polypeptide. According to alternative embodiments, however, the dimeric polypeptide of the invention may comprise a covalent bond located in the HLEM or HLEP portion of the polypeptide linking the monomers.

The dimer is preferably a hetero-dimer. If the polypeptide of the invention is a dimer, each monomer preferably independently comprises an amino acid sequence having a sequence identity of at least 90% to amino acids 764 to 1099, amino acids 764 to 1142, amino acids 764 to 1222, amino acids 764 to 1225, amino acids 764 to 1227 or amino acids 764 to 1242 of SEQ ID NO:18 and is capable of binding to FVIII. In preferred embodiments the VWF moiety in each subunit independently comprises or substantially consists of an amino acid sequence having a sequence identity of at least 95%, at least 96%, at least 97%, at least 98%, or at least 99%, to amino acids 764 to 1099, amino acids 764 to 1142, amino acids 764 to 1222, amino acids 764 to 1225, amino acids 764 to 1227 or amino acids 764 to 1242 of SEQ ID NO:18 and is capable of binding to FVIII. In one embodiment, the VWF moiety of each monomer comprises or substantially consists of amino acids 764 to 1099, amino acids 764 to 1142, amino acids 764 to 1222, amino acids 764 to 1225, amino acids 764 to 1227 or amino acids 764 to 1242 of SEQ ID NO:18.

The VWF moiety may be, or substantially consist of, any one of the VWF fragments disclosed in WO 2013/106787 A1, WO 2014/198699 A2, WO 2011/060242 A2 or WO 2013/093760 A2, the disclosure of which is incorporated herein by reference.

According to further preferred embodiments the VWF moiety as disclosed above may comprise at least one of the amino acid substitutions as disclosed in WO 2016/000039 A1 or WO 2017/117631 A1. Those modified versions of the VWF moiety comprise at least one amino acid substitution within its D′ domain, as compared to the amino acid sequence of the D′ domain of wild-type VWF according to SEQ ID NO:18. The amino acid sequence of the modified versions of the VWF moiety can have one or more amino acid substitutions relative to the respective wild type sequence. The amino acid sequence of the D′ domain of the modified VWF moiety preferably has one or two or three amino acid substitutions relative to the D′ domain of SEQ ID NO:18. It is preferred that S at position 764 of SEQ ID NO:18, corresponding to position 1 of SEQ ID NO:20, is substituted with an amino acid selected from the group consisting of G, P, V, E, Y, A and L. It is also preferred that S at position 766 of SEQ ID NO:18, corresponding to position 3 of SEQ ID NO:20 is substituted with an amino acid selected from the group consisting of Y, I, M, V, F, H, R and W. It is further preferred that V at position 1083 of SEQ ID NO:18 is substituted with the amino acid alanine (A). Preferred combinations of substitutions include S764G/S766Y, S764P/S766I, S764P/S766M, S764V/S766Y, S764E/S766Y, S764Y/S766Y, S764L/S766Y, S764P/S766W, S766W/S806A, S766Y/P769K, S766Y/P769N, S766Y/P769R, S764P/S766L, S764G/S766Y/V1083A (GYA), S764E/S766Y/V1083A (EYA) and S766Y/V1083A (YA), referring to the sequence of SEQ ID NO:18. Most preferred is the combination of substitutions S764E/S766Y/V1083A (EYA), referring to the sequence of SEQ ID NO:18.

The binding affinity of the polypeptide of the present invention to FVIII may be further increased by introduction of said substitutions compared to the binding affinity of a reference polypeptide, which has the same amino acid sequence except for said modifications. Said substitutions within the VWF moiety may contribute to increase the half-life of co-administered FVIII, or the stability of co-formulated FVIII.

Erythrocyte-Binding Moiety

The terms “erythrocyte”, “red blood cell” and “RBC” have the same meaning and are used interchangeably herein.

The erythrocyte-binding moiety is preferably a peptide or polypeptide that is capable of binding to a molecule exposed on the surface of erythrocytes, preferably human erythrocytes. Preferably, the molecule exposed on the surface of erythrocytes is a membrane protein on the surface of erythrocytes, preferably human erythrocytes. More preferably the molecule exposed on the surface of erythrocytes, preferably human erythrocytes, is selected from the group consisting of Band 3 (CD233), aquaporin-1, Glut-1, Kidd antigen, RhAg/Rh50 (CD241), Rh (CD240), Rh30CE (CD240CE), Rh30D (CD240D), Kx, glycophorin A (CD235a), glycophorin B (CD235b), glycophorin C (CD235c), glycophorin D (CD235d), Keil (CD238), Duffy/DARCi (CD234), CR1 (CD35), DAF (CD55), Globoside, CD44, ICAM-4 (CD242), Lu/B-CAM (CD239), XG1/XG2 (CD99), EMMPRIN/neurothelin (CD147), JMH, Glycosyltransferase, Cartwright, Dombrock, C4A/CAB, Scimma, MER2, stomatin, BA-I (CD24), GPIV (CD36), CD108, CD139, and Hantigen (CD173).

Preferably, the erythrocyte-binding moiety is a peptide or polypeptide that is capable of specifically binding to a molecule selected from the group consisting of glycophorin A (CD235a), glycophorin B (CD235b), glycophorin C (CD235c), and glycophorin D (CD235d). Most preferably the erythrocyte-binding moiety capable of specifically binding to glycophorin A (CD235a).

Suitable erythrocyte-binding moieties are described in WO 2019/075523 A1, WO 2014/135528 A1, WO 2018/093766 A1, WO 2013/121296 A1 and WO 2012/021512 A2, the disclosure of which is incorporated herein in its entirety. Examples of erythrocyte-binding moieties binding to human erythrocytes include polypeptides comprising, or essentially consisting of, SEQ ID NO:21 or SEQ ID NO:23. SEQ ID NO:21 shows an exemplary amino acid sequence of a VHH nanobody designated IH4 as disclosed in WO 2014/135528 A1. SEQ ID NO:23 represents a scFv 1C3 obtainable from hybridoma G26.4.1C3/86 [RAT 1C3/86] (ATCC® HB-9893™).

Suitable erythrocyte-binding moieties can be provided by phage display (as described, e.g., in WO 2018/093766 A1 and hybridoma technology.

Preferably, the polypeptide of the invention is a fusion protein, wherein the VWF moiety and the erythrocyte-binding moiety are fused, optionally via a linker sequence.

Half-Life Extending Moiety (HLEM)

In addition to the VWF moiety and the erythrocyte-binding moiety, the polypeptide of the invention may in certain preferred embodiments further comprise a half-life extending moiety. The half-life-extending moiety may be a heterologous amino acid sequence fused to the VWF moiety. Alternatively, the half-life-extending moiety may be chemically conjugated to the polypeptide comprising the VWF moiety by a covalent bond different from a peptide bond.

Preferably, the half-life extending moiety does not induce dimerization or multimerization. Preferably, the half-life extending moiety is not capable of forming dimers or multimers.

In certain embodiments of the invention, the half-life of the polypeptide of the invention is extended by chemical modification, e.g. attachment of a half-life extending moiety such as polyethylene glycol (PEGylation), glycosylated PEG, hydroxyl ethyl starch (HESylation), polysialic acids, elastin-like polypeptides, heparosan polymers or hyaluronic acid. In another embodiment, the polypeptide of the invention is conjugated to a HLEM such as albumin via a chemical linker. The principle of this conjugation technology has been described in an exemplary manner by Conjuchem LLC (see, e.g., U.S. Pat. No. 7,256,253).

In other embodiments, the half-life-extending moiety is a half-life enhancing polypeptide (HLEP). Preferably, the HLEP is an albumin or a fragment thereof. The N-terminus of the albumin may be fused to the C-terminus of the VWF moiety. Alternatively, the C-terminus of the albumin may be fused to the N-terminus of the VWF moiety. One or more HLEPs may be fused to the N- or C-terminal part of the VWF moiety provided that they do not to interfere with or abolish the binding capability of the VWF moiety to FVIII.

The recombinant polypeptide further comprises preferably a covalent bond positioned between the VWF moiety and the HLEM, or a linker sequence positioned between the VWF moiety and the HLEP.

Said linker sequence may be a peptidic linker consisting of one or more amino acids, in particular of 1 to 50, 1 to 30, 1 to 20, 1 to 15, 1 to 10, 1 to 5 or 1 to 3 (e.g. 1, 2 or 3) amino acids and which may be equal or different from each other. Preferably, the linker sequence is not present at the corresponding position in the wild-type VWF. Preferred amino acids present in said linker sequence include Gly and Ser. The linker sequence should be non-immunogenic. Preferred linkers may be comprised of alternating glycine and serine residues. Suitable linkers are described for example in WO 2007/090584 A1.

In another embodiment of the invention the peptidic linker between the VWF moiety and the HLEP consists of peptide sequences, which serve as natural interdomain linkers or sequences in human proteins. Preferably, such peptide sequences in their natural environment are located close to the protein surface and are accessible to the immune system so that one can assume a natural tolerance against this sequence. Examples are given in WO 2007/090584 A1. Cleavable linker sequences are described, e.g., in WO 2013/120939 A1.

In a preferred embodiment of the recombinant polypeptide the linker between the VWF moiety and the HLEP is a glycine/serine peptidic linker having or consisting of amino acid sequence 480 to 510 of SEQ ID NO:2.

In one embodiment the polypeptide has the following structure:

VWFM-L1-H-L2-EBM, [formula 1]

wherein VWFM is the VWF moiety, L1 is a chemical bond or a linker sequence, H is a HLEM, in particular a HLEP, L2 is a chemical bond or a linker sequence and EBM is the erythrocyte-binding moiety.

L1 and L2 independently may be a chemical bond or a linker sequence consisting of one or more amino acids, e.g. of 1 to 50, 1 to 30, 1 to 20, 1 to 15, 1 to 10, 1 to 5 or 1 to 3 (e.g. 1, 2 or 3) amino acids and which may be equal or different from each other. Usually, the linker sequences are not present at the corresponding position in the wild-type VWF. Examples of suitable amino acids present in L1 and/or L2 include Gly and Ser. The linker should be non-immunogenic and may be a non-cleavable or cleavable linker. Non-cleavable linkers may be comprised of alternating glycine and serine residues as exemplified in WO 2007/090584 A1. In another embodiment of the invention the peptidic linker between the VWF moiety and the albumin moiety consists of peptide sequences, which serve as natural interdomain linkers or sequences in human proteins. Preferably such peptide sequences in their natural environment are located close to the protein surface and are accessible to the immune system so that one can assume a natural tolerance against this sequence. Examples are given in WO 2007/090584 A1. Cleavable linker sequences are described, e.g. in WO 2013/120939 A1.

Preferred HLEP sequences are described infra. Likewise encompassed by the invention are fusions to the exact “N-terminal amino acid” or to the exact “C-terminal amino acid” of the respective HLEP, or fusions to the “N-terminal part” or “C-terminal part” of the respective HLEP, which includes N-terminal deletions of one or more amino acids of the HLEP. The polypeptide may comprise more than one HLEP sequence, e.g. two or three HLEP sequences. These multiple HLEP sequences may be fused to the C-terminal part of VWF in tandem, e.g. as successive repeats.

Half-Life Enhancing Polypeptides (HLEPs)

Preferably, the half-life extending moiety is a half-life enhancing polypeptide (HLEP). More preferably the HLEP is selected from the group consisting of albumin, a member of the albumin-family or fragments thereof, solvated random chains with large hydrodynamic volume (e.g. XTEN (Schellenberger et al. 2009; Nature Biotechnol. 27:1186-1190), homo-amino acid repeats (HAP) or proline-alanine-serine repeats (PAS), afamin, alpha-fetoprotein, Vitamin D binding protein, transferrin or variants or fragments thereof, carboxyl-terminal peptide (CTP) of human chorionic gonadotropin-β subunit, a polypeptide capable of binding to the neonatal Fc receptor (FcRn), in particular an immunoglobulin constant region and portions thereof, e.g. the Fc fragment, polypeptides or lipids capable of binding under physiological conditions to albumin, to a member of the albumin-family or to fragments thereof or to an immunoglobulin constant region or portions thereof. The immunoglobulin constant region or portions thereof is preferably an Fc fragment of immunoglobulin G1, an Fc fragment of immunoglobulin G2 or an Fc fragment of immunoglobulin A.

Preferably, the HLEP does not induce dimerization or multimerization. Preferably, the HLEP is not capable of forming dimers or multimers.

A half-life enhancing polypeptide as used herein may be a full-length half-life enhancing protein described herein or one or more fragments thereof that are capable of stabilizing or prolonging the therapeutic activity or the biological activity of the coagulation factor, in particular of increasing the in vivo half-life of the polypeptide of the invention. Such fragments may be of 10 or more amino acids in length or may include at least about 15, at least about 20, at least about 25, at least about 30, at least about 50, at least about 100, or more contiguous amino acids from the HLEP sequence or may include part or all of specific domains of the respective HLEP, as long as the HLEP fragment provides a functional half-life extension of at least 25% compared to the respective polypeptide without the HLEP.

The HLEP portion of the polypeptide of the invention may be a variant of a wild type HLEP. The term “variants” includes insertions, deletions and substitutions, either conservative or non-conservative, where such changes do not substantially alter the FVIII-binding activity of the VWF moiety.

In particular, the proposed VWF moiety-HLEP fusion constructs of the invention may include naturally occurring polymorphic variants of HLEPs and fragments of HLEPs. The HLEP may be derived from any vertebrate, especially any mammal, for example human, monkey, cow, sheep, or pig. Non-mammalian HLEPs include, but are not limited to, hen and salmon.

According to certain embodiments of the present disclosure, the HLEM, in particular a HLEP, portion of the recombinant polypeptide of the invention may be specified with the alternative term “FP”. Preferably, the term “FP” represents a human albumin.

According to certain preferred embodiments, the recombinant polypeptide is a fusion protein. A fusion protein in terms of the present invention is a protein created by in-frame joining of at least two DNA sequences encoding the VWF moiety as well as the HLEP. The skilled person understands that translation of the fusion protein DNA sequence will result in a single protein sequence. As a result of an in frame insertion of a DNA sequence encoding a peptidic linker according to a further preferred embodiment, a fusion protein comprising the VWF moiety, a suitable linker and the HLEP may be obtained.

According to some embodiments, the co-formulated FVIII does neither comprise any of the herein described HLEM or HLEP structures. According to certain other embodiments, the co-formulated FVIII may comprise at least one of the herein described HLEM or HLEP structures.

Albumin as HLEP

The terms, “human serum albumin” (HSA) and “human albumin” (HA) are used interchangeably in this application. The terms “albumin” and “serum albumin” are broader, and encompass human serum albumin (and fragments and variants thereof) as well as albumin from other species (and fragments and variants thereof).

As used herein, “albumin” refers collectively to albumin polypeptide or amino acid sequence, or an albumin fragment or variant, having one or more functional properties (e.g., biological functions) of albumin. In particular, “albumin” refers to human albumin or fragments thereof, especially the mature form of human albumin as shown in SEQ ID NO:19 herein or albumin from other vertebrates or fragments thereof, or analogs or variants of these molecules or fragments thereof.

According to certain embodiments of the present disclosure the alternative term “FP” is used to identify the HLEP, in particular to define albumin as HLEP.

In particular, the proposed polypeptides of the invention may include naturally occurring polymorphic variants of human albumin and fragments of human albumin. Generally speaking, an albumin fragment or variant will be at least 10, preferably at least 40, most preferably more than 70 amino acids long.

Preferred embodiments of the invention include albumin variants used as a HLEP of the polypeptide of the invention with enhanced binding to the FcRn receptor. Such albumin variants may lead to a longer plasma half-life of a VWF moiety albumin variant fusion protein as compared to a VWF moiety fusion with a wild-type albumin.

The albumin portion of the polypeptides of the invention may comprise at least one subdomain or domain of HA or conservative modifications thereof.

Immunoglobulins as HLEPs

Immunoglobulin G (IgG) constant regions (Fc) are known in the art to increase the half-life of therapeutic proteins (Dumont J A et al. 2006. BioDrugs 20:151-160). The IgG constant region of the heavy chain consists of three domains (CH1-CH3) and a hinge region. The immunoglobulin sequence may be derived from any mammal, or from subclasses IgG 1, IgG2, IgG3 or IgG4, respectively. IgG and IgG fragments without an antigen-binding domain may also be used as HLEPs. The therapeutic polypeptide portion is connected to the IgG or the IgG fragments preferably via the hinge region of the antibody or a peptidic linker, which may even be cleavable. Several patents and patent applications describe the fusion of therapeutic proteins to immunoglobulin constant regions to enhance the therapeutic proteins' in vivo half-lives. US 2004/0087778 and WO 2005/001025 A2 describe fusion proteins of Fc domains or at least portions of immunoglobulin constant regions with biologically active peptides that increase the half-life of the peptide, which otherwise would be quickly eliminated in vivo. Fc-IFN-β fusion proteins were described that achieved enhanced biological activity, prolonged circulating half-life and greater solubility (WO 2006/000448 A2). Fc-EPO proteins with a prolonged serum half-life and increased in vivo potency were disclosed (WO 2005/063808 A1) as well as Fc fusions with G-CSF (WO 2003/076567 A2), glucagon-like peptide-1 (WO 2005/000892 A2), clotting factors (WO 2004/101740 A2) and interleukin-10 (U.S. Pat. No. 6,403,077), all with half-life enhancing properties.

Preferably, the immunoglobulin or Fc portion to be used as HLEP does not induce dimerization or multimerization. Preferably, the immunoglobulin or Fc portion to be used as HLEP is not capable of forming dimers or multimers.

Various HLEPs which can be used in accordance with this invention are described in detail in WO 2013/120939 A1.

Dimers

The polypeptides of this invention may have a high proportion of dimers. The polypeptide of the invention is therefore preferably present as dimer. In one embodiment, at least 50%, or at least 60%, or at least 70%, or at least 80%, or at least 90%, or at least 95% or at least 98% of the polypeptides are present as dimers. Most preferably, essentially all polypeptides of the invention are present as dimers. The dimer is preferably a heterodimer.

Further preferred is that the polypeptide of the invention does not comprise multimeric forms. The use of dimers is favorable, as the dimer has an improved affinity to Factor VIII as compared to the monomer. The dimer content and the ratio of dimer to monomer of the polypeptide of the invention can be determined by size exclusion chromatography or HPLC, e.g. as described on page 56, lines 6-10 of WO 2010/087271 A1. Alternatively, the dimer content and the ratio of dimer to monomer can be determined by SDS-PAGE and Western Blot, see examples of the present application.

Unless specifically indicated otherwise, any molar concentration of the polypeptide of the invention described herein refers to the molar concentration of the dimer of the polypeptide of the invention, whether actually present as homodimer or heterodimer.

In one embodiment, the affinity of the polypeptide of the invention to Factor VIII is greater than that of human native VWF to the same Factor VIII molecule. The Factor VIII affinity of the polypeptide may refer to human native, either plasma-derived or recombinant, Factor VIII, in particular to a recombinant Factor VIII molecule having a truncated or deleted B-domain.

It has been found that preparations of the polypeptide of this invention with a high proportion of dimers do have an increased affinity to Factor VIII. Alternatively to, or in combination with an increased dimer proportion also polypeptides in accordance with the invention with mutations within the Factor VIII binding domain which do increase the affinity to Factor VIII are preferred embodiments of the invention. Suitable mutations are disclosed, e.g., in WO 2013/120939 A1.

Preferably, the polypeptide of the invention is a heterodimer. In one embodiment the heterodimer comprises a first subunit and a second subunit, wherein the first subunit comprises a first VWF moiety as defined herein and the erythrocyte-binding moiety, and the second subunit comprises a second VWF moiety as defined herein, wherein the second subunit does not comprise an erythrocyte-binding moiety. The VWF moieties in the first and second subunit (monomer) are preferably identical.

In one embodiment, the second subunit substantially consists of the second VWF moiety.

In another embodiment, the two monomers forming the dimer are covalently linked to each other via at least one disulfide bridge formed by cysteine residues within the VWF moiety. The cysteine residues forming the one or more disulfide bridges may be selected from the group consisting of Cys-1099, Cys-1142, Cys-1222, Cys-1225, Cys-1227 and combinations thereof, preferably Cys-1099 and Cys-1142, wherein the amino acid numbering refers to SEQ ID NO:18.

Preparation of the Polypeptide

The nucleic acid encoding the polypeptide of the invention can be prepared according to methods known in the art. Based on the cDNA sequence of pre-pro form of human native VWF (SEQ ID NO:17), recombinant DNA encoding the above-mentioned VWF moiety constructs or polypeptides of the invention can be designed and generated.

Even if the polypeptide which is secreted by the host cells does not comprise amino acids 1 to 763 of pre-pro form of human VWF, it is preferred that the nucleic acid (e.g. the DNA) encoding the intracellular precursor of the polypeptide comprises a nucleotide sequence encoding an amino acid sequence having a sequence identity of at least 95%, at least 96%, at least 97%, at least 98%, or at least 99%, to amino acids 23 to 763 or preferably to amino acids 1 to 763 of SEQ ID NO:18. Most preferably, the nucleic acid (e.g. the DNA) encoding the intracellular precursor of the polypeptide comprises a nucleotide sequence encoding amino acids 23 to 763 of SEQ ID NO:18, or amino acids 1 to 763 of SEQ ID NO:18.

Constructs in which the DNA contains the entire open reading frame inserted in the correct orientation into an expression plasmid may be used for protein expression. Typical expression vectors contain promoters that direct the synthesis of large amounts of mRNA corresponding to the inserted nucleic acid in the plasmid-bearing cells. They may also include an origin of replication sequence allowing for their autonomous replication within the host organism, and sequences that increase the efficiency with which the synthesized mRNA is translated. Stable long-term vectors may be maintained as freely replicating entities by using regulatory elements of, for example, viruses (e.g., the OriP sequences from the Epstein Barr Virus genome). Cell lines may also be produced that have integrated the vector into the genomic DNA, and in this manner the gene product is produced on a continuous basis.

Typically, the cells to be provided are obtained by introducing the nucleic acid encoding a polypeptide of the invention into mammalian host cells.

Any host cell susceptible to cell culture, and to expression of glycoproteins, may be utilized in accordance with the present invention. In certain embodiments, a host cell is mammalian. Non-limiting examples of mammalian cells that may be used in accordance with the present invention include BALB/c mouse myeloma line (NSO/1, ECACC No: 85110503); human retinoblasts (PER.C6 (CruCell, Leiden, The Netherlands)); monkey kidney CV1 line transformed by SV40 (COS-7, ATCC CRL 1651); human embryonic kidney line (293 or 293 cells subcloned for growth in suspension culture, Graham et al., J. Gen Virol., 36:59, 1977); baby hamster kidney cells (BHK, ATCC CCL10); Chinese hamster ovary cells+/−DHFR (CHO, Urlaub and Chasin, Proc. Natl. Acad. Sci. USA, 77:4216, 1980); mouse sertoli cells (TM4, Mather, Biol. Reprod., 23:243 251, 1980); monkey kidney cells (CV1 ATCC CCL 70); African green monkey kidney cells (VERO-76, ATCC CRL-1 587); human cervical carcinoma cells (HeLa, ATCC CCL 2); canine kidney cells (MDCK, ATCC CCL 34); buffalo rat liver cells (BRL 3A, ATCC CRL 1442); human lung cells (W138, ATCC CCL 75); human liver cells (HepG2, HB 8065); mouse mammary tumor (MMT 060562, ATCC CCL51); TRI cells (Mather et al., Annals NY. Acad. Sci., 383:44-68, 1982); MRC 5 cells; PS4 cells; human amniocyte cells (CAP); and a human hepatoma line (Hep G2). Preferably, the cell line is a rodent cell line, especially a hamster cell line such as CHO or BHK or a human cell line.

Methods suitable for introducing nucleic acids sufficient to achieve expression of a glycoprotein of interest into mammalian host cells are known in the art. See, for example, Gething et al., Nature, 293:620-625, 1981; Mantei et al., Nature, 281:40-46, 1979; Levinson et al. EP 0117060; and EP 0117058. For mammalian cells, common methods of introducing genetic material into mammalian cells include the calcium phosphate precipitation method of Graham and van der Erb (Virology, 52:456-457, 1978) or the Lipofectamine™ (Gibco BRL) Method of Hawley-Nelson (Focus 15:73, 1993). General aspects of mammalian cell host system transformations have been described by Axel in U.S. Pat. No. 4,399,216. For various techniques for introducing genetic material into mammalian cells, see Keown et al., Methods in Enzymology, 185:527-537, 1990, and Mansour et al., Nature, 336:348-352, 1988.

The cells are cultured under conditions that allow expression of the polypeptide. The polypeptide can be recovered and purified using methods that are known to the skilled artisan.

Therapeutic Use

The polypeptides of the invention are useful for treating coagulation disorders including hemophilia A. The term “hemophilia A” refers to a deficiency in functional coagulation FVIII, which is usually inherited.

A further aspect of this invention is a method of treating a blood coagulation disorder, comprising administering to a patient in need thereof an effective amount of a polypeptide as defined hereinabove.

Treatment of a disease encompasses the treatment of patients already diagnosed as having any form of the disease at any clinical stage or manifestation; the delay of the onset or evolution or aggravation or deterioration of the symptoms or signs of the disease; and/or preventing and/or reducing the severity of the disease.

A “subject” or “patient” to whom a polypeptide of the invention is administered preferably is a human. In certain aspects, the human is a pediatric patient. In other aspects, the human is an adult patient.

Compositions comprising a polypeptide of the invention are described herein. The compositions typically are supplied as part of a sterile, pharmaceutical composition that includes a pharmaceutically acceptable carrier. This composition can be in any suitable form (depending upon the desired method of administering it to a patient).

The polypeptide of the invention can be administered to a patient by a variety of extravascular routes such as subcutaneously, intradermally or intramuscularly. The most suitable route for administration in any given case will depend on the particular polypeptide, the subject, and the nature and severity of the disease and the physical condition of the subject. Preferably, a polypeptide of the invention will be administered subcutaneously.

In one embodiment, the treatment comprises administering the polypeptide of the invention as the sole active ingredient. In another embodiment, the treatment comprises administering the polypeptide of the invention in combination with at least one further active ingredient. The polypeptide of the invention and the at least one further active ingredient can be administered simultaneously, separately or sequentially.

Preferably, the further active ingredient is FVIII. The method of the invention therefore preferably comprises administering to the patient an effective amount of FVIII. The polypeptide of the invention and the FVIII are preferably co-administered subcutaneously.

Determination of the total number of doses and length of treatment with a polypeptide of the invention and FVIII is well within the capabilities of those skilled in the art. The dosage of the polypeptide of the invention as well as FVIII to be administered depends on the concentrations of the FVIII to be administered. The degree of severity of the blood coagulation disorder may also be considered to determine the appropriate dosage of the polypeptide of the invention as well as of FVIII to be administered. Typical dosages for FVIII may range from about 20 IU/kg body weight to about 1000 IU/kg body weight, preferably from about 20 IU/kg body weight to about 500 IU/kg body weight, further preferred from about 20 IU/kg body weight to about 400 IU/kg body weight, more preferred from about 20 IU/kg body weight to about 300 IU/kg body weight.

In accordance with this invention, the patient being treated with the polypeptide of the invention is also treated with blood coagulation Factor VIII. The polypeptide of the invention and the Factor VIII may preferably be administered simultaneously, i.e. together, although an administration in a sequential fashion could in principle also be performed, both modes of administration being encompassed by the term “combination therapy” and “co-administration”. The polypeptide of the invention and the Factor VIII may be administered as a mixture, i.e. within the same composition, or separately, i.e. as separate compositions. Co-administration of the recombinant polypeptide and the FVIII protein is preferably achieved by administration together in a single composition comprising the recombinant polypeptide and the FVIII protein. According to further preferred embodiments, co-administration of the recombinant polypeptide and the FVIII protein is achieved by providing a combination product comprising the recombinant polypeptide and the FVIII blended in a single composition or by providing a set or kit of at least two separate products arranged to be mixed before administration, whereby a first product comprises the recombinant polypeptide and a second product comprises the FVIII. In particular, in case that the recombinant polypeptide and the FVIII protein are provided in separate compositions or products to be mixed prior to co-administration, the mixture may be treated before administration in such a manner to allow prior to administration for at least a proportion of said recombinant polypeptide to bind to said FVIII. For example, the mixture could be incubated for a certain time. Such incubation could be conducted in less than 1 min, or less than 5 min at either ambient temperature or, if appropriate, at elevated temperature, however, preferably at a temperature below 40° C. Such a quick incubation step may also be appropriate during reconstitution for a combination product comprising the recombinant polypeptide and the FVIII blended in a single composition.

The concentration of Factor VIII in the composition used is typically in the range of 10-10,000 IU/mL. In different embodiments, the concentration of FVIII in the compositions of the invention is in the range of 10-8,000 IU/mL, or 10-5,000 IU/mL, or 20-3,000 IU/mL, or 50-1,500 IU/mL, or 3,000 IU/mL, or 2,500 IU/mL, or 2,000 IU/mL, or 1,500 IU/mL, or 1,200 IU/mL, or 1,000 IU/mL, or 800 IU/mL, or 750 IU/mL, or 600 IU/mL, or 500 IU/mL, or 400 IU/mL, or 300 IU/mL, or 250 IU/mL, or 200 IU/mL, or 150 IU/mL, or 125 IU/mL, or 100 IU/mL, or 62.5 IU/mL, or 50 IU/mL, provided the requirements regarding the ratio with respect to the VWF polypeptide of the invention as defined herein are fulfilled.

“International Unit,” or “IU,” is a unit of measurement of the blood coagulation activity (potency) of FVIII as measured by a FVIII activity assay such as a one stage clotting assay or a chromogenic substrate FVIII activity assay using a standard calibrated in “IU” against an international standard preparation. One stage clotting assays are known to the art, such as that described in N Lee, Martin L, et al., An Effect of Predilution on Potency Assays of FVIII Concentrates, Thrombosis Research (Pergamon Press Ltd.) 30, 511 519 (1983). Principle of the one stage assay: The test is executed as a modified version of the activated Partial Thromboplastin Time (aPTT)-assay: Incubation of plasma with phospholipids and a surface activator leads to the activation of factors of the intrinsic coagulation system. Addition of calcium ions triggers the coagulation cascade. The time to formation of a measurable fibrin clot is determined. The assay is executed in the presence of Factor VIII deficient plasma. The coagulation capability of the deficient plasma is restored by Coagulation Factor VIII included in the sample to be tested. The shortening of coagulation time is proportional to the amount of Factor VIII present in the sample. The activity of Coagulation Factor VIII is quantified by direct comparison to a standard preparation with a known activity of Factor VIII in International Units. Another standard assay is a chromogenic substrate assay. Chromogenic substrate assays may be purchased commercially, such as the Coamatic® FVIII test kit (Chromogenix-Instrumentation Laboratory SpA V. le Monza 338-20128 Milano, Italy). Principle of the chromogenic assay: In the presence of calcium and phospholipid, Factor X is activated by Factor IXa to Factor Xa. This reaction is stimulated by Factor Villa as cofactor. FVIIIa is formed by low amounts of thrombin in the reaction mixture from FVIII in the sample to be measured.

When using the optimum concentrations of Ca2+, phospholipid and Factor IXa and an excess quantity of Factor X, activation of Factor X is proportional to the potency of Factor VIII. Activated Factor X releases the chromophore pNA from the chromogenic substrate S-2765. The release of pNA, measured at 405 nm, is therefore proportional to the amount of FXa formed, and, therefore, also to the Factor VIII activity of the sample.

If the polypeptide of the invention and FVIII are used in a combination therapy, the ratio of polypeptide to FVIII to be administered can be any ratio as defined infra in the section “Ratios”. In another aspect the present invention relates to the polypeptide of the present invention for use in increasing the half-life of FVIII in vivo. Yet another aspect of the invention is a method of increasing the half-life of FVIII in a subject, comprising administering to the subject an effective amount of the polypeptide of the present invention, or of the pharmaceutical composition of the present invention. The polypeptide of the invention and the FVIII may be co-administered to the subject simultaneously, separately or sequentially.

In yet another aspect the present invention relates the polypeptide of the present invention for use in preventing and/or reducing inhibitor formation in vivo. Yet another aspect of the invention is a method of reducing and/or preventing inhibitor formation in a subject being treated with FVIII, said method comprising administering to the subject an effective amount of the polypeptide of the invention.

Factor VIII

As used herein, the term “Factor VIII” or “FVIII” refers to molecules having at least part of the coagulation activity of human native Factor VIII. Human FVIII consists of 2351 amino acids (including a signal peptide) and 2332 amino acids (without the signal peptide). “Human native FVIII” is the human plasma-derived FVIII molecule having the full length sequence as shown in SEQ ID NO:20 (amino acid 1-2332). The detailed domain structure, A1-a1-A2-a2-B-a3-A3-C1-C2 has the corresponding amino acid residues (referring to SEQ ID NO:20): A1 (1-336), a1 (337-372), A2 (373-710), a2 (711-740), B (741-1648), a3 (1649-1689), A3 (1690-2020), C1 (2021-2173) and C2 (2174-2332). The FVIII referred to herein may be a plasma-derived FVIII (pdFVIII) or a recombinantly produced FVIII (recombinant FVIII).

The coagulation activity of the FVIII molecule can be determined using a one-stage clotting assay (e.g. as described in Lee et al., Thrombosis Research 30, 511 519 (1983)) or a chromogenic substrate assay (e.g. the coamatic FVIII test kit from Chromogenix-Instrumentation Laboratory SpA V. le Monza 338-20128 Milano, Italy). Further details of these activity assays are described infra.

Preferably, the FVIII molecules used in accordance with this invention have at least 10% of the specific molar activity of human native FVIII. The term “specific molar activity” refers to the coagulation activity per mole of FVIII and is indicated e.g. in “IU/mole” FVIII or—more convenient—in “IU/pmole” FVIII.

In a preferred embodiment, the FVIII molecule is a non-naturally occurring FVIII molecule. Preferably, the non-naturally occurring FVIII molecule has been produced recombinantly. In another embodiment, the FVIII molecule has been produced in cell culture. In another preferred embodiment, the non-naturally occurring FVIII molecule has a glycosylation pattern different from that of plasma-derived FVIII. In yet another embodiment, the FVIII molecule is selected from the group consisting of (i) B-domain deleted or truncated FVIII molecules, (ii) single-chain FVIII molecules, (iii) recombinantly produced two-chain FVIII molecules, (iv) FVIII molecules having protective groups or half-life extending moieties, (v) fusion proteins comprising a FVIII amino acid sequence fused to a heterologous amino acid sequence, and (vi) combinations thereof.

The terms “Factor VIII” and “FVIII” are used synonymously herein. “Factor VIII compositions” in the sense of the present invention include compositions comprising FVIII and FVIIIa. FVIIIa may typically be present in small amounts, e.g. about 1 to 2% FVIIIa, referred to the total amount of FVIII protein in the composition. Proteolytically cleaved FVIII may typically be present in small to medium amounts, e.g. about 1 to 50%, referred to the total amount of FVIII protein in the composition. “FVIII” includes natural allelic variations of FVIII that may exist and occur from one individual to another. FVIII may be plasma-derived or recombinantly produced, using well-known methods of production and purification. The degree and location of glycosylation, tyrosine sulfation and other post-translation modifications may vary, depending on the chosen host cell and its growth conditions.

The term FVIII includes FVIII analogues. The term “FVIII analogue” as used herein refers to a FVIII molecule (full-length or B-domain-truncated/deleted) wherein one or more amino acids have been substituted or deleted compared to SEQ ID NO:20 or, for B-domain truncated/deleted FVII molecules, the corresponding part of SEQ ID NO:20. FVIII analogues do not occur in nature but are obtained by human manipulation.

The Factor VIII molecules used in accordance with the present invention may also be B-domain-truncated/deleted FVIII molecules wherein the remaining domains correspond to the sequences as set forth in amino acid numbers 1-740 and 1649-2332 of SEQ ID NO:20. Other forms of B-domain deleted FVIII molecules have additionally a partial deletion in their a3 domain, which leads to single-chain FVIII molecules.

It follows that these FVIII molecules are recombinant molecules produced in transformed host cells, preferably of mammalian origin. However, the remaining domains in a B-domain deleted FVIII, (i.e. the three A-domains, the two C-domains and the a1, a2 and a3 regions) may differ slightly e.g. about 1%, 2%, 3%, 4% or 5% from the respective amino acid sequence as set forth in SEQ ID NO:20 (amino acids 1-740 and 1649-2332).

The FVIII molecules used in accordance with the present invention may be two-chain FVIII molecules or single-chain FVIII molecules. The FVIII molecules used in accordance with the present invention may also be biologically active fragments of FVIII, i.e., FVIII wherein domain(s) other than the B-domain has/have been deleted or truncated, but wherein the FVIII molecule in the deleted/truncated form retains its ability to support the formation of a blood clot. FVIII activity can be assessed in vitro using techniques well known in the art. A preferred test for determining FVIII activity according to this invention is the chromogenic substrate assay or the one stage assay (see infra). Amino acid modifications (substitutions, deletions, etc.) may be introduced in the remaining domains, e.g., in order to modify the binding capacity of Factor VIII with various other components such as e.g. Von Willebrand Factor (vWF), low density lipoprotein receptor-related protein (LPR), various receptors, other coagulation factors, cell surfaces, etc. or in order to introduce and/or abolish glycosylation sites, etc. Other mutations that do not abolish FVIII activity may also be accommodated in a FVIII molecule/analogue for use in a composition of the present invention.

FVIII analogues also include FVIII molecules, in which one or more of the amino acid residues of the parent polypeptide have been deleted or substituted with other amino acid residues, and/or wherein additional amino acid residues has been added to the parent FVIII polypeptide.

Furthermore, the Factor VIII molecules/analogues may comprise other modifications in e.g. the truncated B-domain and/or in one or more of the other domains of the molecules (“FVIII derivatives”). These other modifications may be in the form of various molecules conjugated to the Factor VIII molecule, such as e.g. polymeric compounds, peptidic compounds, fatty acid derived compounds, etc.

The term FVIII includes FVIII molecules having protective groups or half-life extending moieties. The terms “protective groups”/“half-life extending moieties” is herein understood to refer to one or more chemical groups attached to one or more amino acid site chain functionalities such as —SH, —OH, —COOH, —CONH2, —NH2, or one or more N- and/or O-glycan structures and that can increase in vivo circulatory half-life of a number of therapeutic proteins/peptides when conjugated to these proteins/peptides. Examples of protective groups/half-life extending moieties include: Biocompatible fatty acids and derivatives thereof, Hydroxy Alkyl Starch (HAS) e.g. Hydroxy Ethyl Starch (HES), Poly (Glyₓ-Ser_{y})ₙ (Homo Amino acid Polymer (HAP)), Hyaluronic acid (HA), Heparosan polymers (HEP), Phosphorylcholine-based polymers (PC polymer), Fleximer® polymers (Mersana Therapeutics, MA, USA), Dextran, Poly-sialic acids (PSA), polyethylene glycol (PEG), an Fc domain, Transferrin, Albumin, Elastin like peptides, XTEN® polymers (Amunix, CA, USA), Albumin binding peptides, a von Willebrand factor fragment (vWF fragment), a Carboxyl Terminal Peptide (CTP peptide, Prolor Biotech, IL), and any combination thereof (see, for example, McCormick, C. L., A. B. Lowe, and N. Ayres, Water-Soluble Polymers, in Encyclopedia of Polymer Science and Technology. 2002, John Wiley & Sons, Inc.). The manner of derivatization is not critical and can be elucidated from the above.

The term FVIII includes glyco-pegylated FVIII. In the present context, the term “glyco-pegylated FVIII” is intended to designate a Factor VIII molecule (including full length FVIII and B-domain truncated/deleted FVIII) wherein one or more PEG group(s) has/have been attached to the FVIII polypeptide via the polysaccharide sidechain(s) (glycan(s)) of the polypeptide.

The FVIII molecules which can be used in accordance with this invention include fusion proteins comprising a FVIII amino acid sequence fused to a heterologous amino acid sequence, preferably a half-life extending amino acid sequence. Preferred fusion proteins are Fc fusion proteins and albumin fusion proteins. The term “Fc fusion protein” is herein meant to encompass FVIII fused to an Fc domain that can be derived from any antibody isotype. An IgG Fc domain will often be preferred due to the relatively long circulatory half-life of IgG antibodies. The Fc domain may furthermore be modified in order to modulate certain effector functions such as e.g. complement binding and/or binding to certain Fc receptors. Fusion of FVIII with an Fc domain, which has the capacity to bind to FcRn receptors, will generally result in a prolonged circulatory half-life of the fusion protein compared to the half-life of the wt FVIII. It follows that a FVIII molecule for use in the present invention may also be a derivative of a FVIII analogue, such as, for example, a fusion protein of an FVIII analogue, a PEGylated or glycoPEGylated FVIII analogue, or a FVIII analogue conjugated to a heparosan polymer. The term “albumin fusion protein” is herein meant to encompass FVIII fused to an albumin amino acid sequence or a fragment or derivative thereof. The heterologous amino acid sequence may be fused to the N- or C-terminus of FVIII, or it may be inserted internally within the FVIII amino acid sequence. The heterologous amino acid sequence may be any “half life extending polypeptide” described in WO 2008/077616 A1, the disclosure of which is incorporated herein by reference.

Examples of FVIII molecules that may be used in accordance with the present invention comprise for instance the FVIII molecules described in WO 2010/045568 A1, WO 2009/062100 A1, WO 2010/014708 A2, WO 2008/082669 A2, WO 2007/126808 A1, US 2010/0173831, US 2010/0173830, US 2010/0168391, US 2010/0113365, US 2010/0113364, WO 2003/031464 A2, WO 2009/108806 A1, WO 2010/102886 A1, WO 2010/115866 A1, WO 2011/101242 A1, WO 2011/101284 A1, WO 2011/101277 A1, WO 2011/131510 A1, WO 2012/007324 A2, WO 2011/101267 A1, WO 2013/083858 A1, and WO 2004/067566 A1.

Examples of FVIII molecules, which can be used in accordance with the present invention include the active ingredient of Advate®, Helixate®, Kogenate®, Xyntha®, Adynovate®, Kovaltry®, Novo8®, Nuwiq®, Novoeight®, Eloctate®, as well as the FVIII molecule described in WO 2008/135501 A1, WO 2009/007451 A1 and the construct designated “dBN(64-53)” of WO 2004/067566 A1. This construct has the amino acid sequence shown in SEQ ID NO 5.

“International Unit,” or “IU,” is a unit of measurement of the blood coagulation activity (potency) of FVIII as measured by a FVIII activity assay such as a one stage clotting assay or a chromogenic substrate FVIII activity assay using a standard calibrated against an international standard preparation calibrated in “IU”. One stage clotting assays are known to the art, such as that described in N Lee, Martin L, et al., An Effect of Predilution on Potency Assays of FVIII Concentrates, Thrombosis Research (Pergamon Press Ltd.) 30, 511 519 (1983). Principle of the one stage assay: The test is executed as a modified version of the activated Partial Thromboplastin Time (aPTT)-assay: Incubation of plasma with phospholipids and a surface activator leads to the activation of factors of the intrinsic coagulation system. Addition of calcium ions triggers the coagulation cascade. The time to formation of a measurable fibrin clot is determined. The assay is executed in the presence of Factor VIII deficient plasma. The coagulation capability of the deficient plasma is restored by Coagulation Factor VIII included in the sample to be tested. The shortening of coagulation time is proportional to the amount of Factor VIII present in the sample. The activity of Coagulation Factor VIII is quantified by direct comparison to a standard preparation with a known activity of Factor VIII in International Units.

Another standard assay is a chromogenic substrate assay. Chromogenic substrate assays may be purchased commercially, such as the coamatic FVIII test kit (Chromogenix-Instrumentation Laboratory SpA V. le Monza 338-20128 Milano, Italy). Principle of the chromogenic assay: In the presence of calcium and phospholipid, Factor X is activated by Factor IXa to Factor Xa. This reaction is stimulated by Factor Villa as cofactor. FVIIIa is formed by low amounts of thrombin in the reaction mixture from FVIII in the sample to be measured. When using the optimum concentrations of Ca²⁺, phospholipid and Factor IXa and an excess quantity of Factor X, activation of Factor X is proportional to the potency of Factor VIII. Activated Factor X releases the chromophore pNA from the chromogenic substrate S-2765. The release of pNA, measured at 405 nm, is therefore proportional to the amount of FXa formed, and, therefore, also to the Factor VIII activity of the sample.

Pharmaceutical Compositions

Another aspect of the present invention is a pharmaceutical composition comprising the polypeptide of the invention and optionally a pharmaceutically acceptable carrier, diluent or excipient. In a first embodiment the pharmaceutical composition comprises the polypeptide of the invention as the sole active ingredient. In a second embodiment, the pharmaceutical composition comprises the polypeptide of the invention and at least one further active ingredient. Preferably, the further active ingredient is a FVIII molecule as described supra. The ratio of the polypeptide of the invention to the FVIII in the pharmaceutical composition can be any ratio as defined in the section “Ratios” infra.

Therapeutic formulations of the polypeptide of the invention can be prepared for storage as lyophilized formulations or aqueous solutions by mixing the polypeptide of the invention having the desired degree of purity with optional pharmaceutically-acceptable carriers, excipients or stabilizers typically employed in the art (all of which are referred to herein as “carriers”), i.e., buffering agents, stabilizing agents, preservatives, isotonifiers, non-ionic detergents, antioxidants, and other miscellaneous additives. See, Remington's Pharmaceutical Sciences, 16th edition (Osol, ed. 1980). Such additives must be nontoxic to the recipients at the dosages and concentrations employed.

Buffering agents help to maintain the pH in the range, which approximates physiologically acceptable conditions. They can be present at concentrations ranging typically from about 2 mM to about 100 mM. Suitable buffering agents include both organic and inorganic acids and salts thereof such as citrate buffers (e.g., monosodium citrate-disodium citrate mixture, citric acid-trisodium citrate mixture, citric acid-monosodium citrate mixture, etc.), succinate buffers (e.g., succinic acid-monosodium succinate mixture, succinic acid-sodium hydroxide mixture, succinic acid-disodium succinate mixture, etc.), tartrate buffers (e.g., tartaric acid-sodium tartrate mixture, tartaric acid-potassium tartrate mixture, tartaric acid-sodium hydroxide mixture, etc.), fumarate buffers (e.g., fumaric acid-monosodium fumarate mixture, fumaric acid-disodium fumarate mixture, monosodium fumarate-disodium fumarate mixture, etc.), gluconate buffers (e.g., gluconic acid-sodium glyconate mixture, gluconic acid-sodium hydroxide mixture, gluconic acid-potassium gluconate mixture, etc.), oxalate buffer (e.g., oxalic acid-sodium oxalate mixture, oxalic acid-sodium hydroxide mixture, oxalic acid-potassium oxalate mixture, etc), lactate buffers (e.g., lactic acid-sodium lactate mixture, lactic acid-sodium hydroxide mixture, lactic acid-potassium lactate mixture, etc.) and acetate buffers (e.g., acetic acid-sodium acetate mixture, acetic acid-sodium hydroxide mixture, etc.). Additionally, phosphate buffers, histidine buffers and trimethylamine salts such as Tris can be used.

Preservatives can be added to retard microbial growth, and can be added typically in amounts ranging from 0.2%-1% (w/v). Suitable preservatives include phenol, benzyl alcohol, meta-cresol, methyl paraben, propyl paraben, octadecyldimethylbenzyl ammonium chloride, benzalconium halides (e.g., chloride, bromide, and iodide), hexamethonium chloride, and alkyl parabens such as methyl or propyl paraben, catechol, resorcinol, cyclohexanol, and 3-pentanol.

Tonicity modifying agents, sometimes known as “stabilizers”, can be added to ensure a pharmaceutically acceptable tonicity, preferably isotonicity, of liquid compositions and include polyhydric sugar alcohols, preferably trihydric or higher sugar alcohols, such as glycerin, erythritol, arabitol, xylitol, sorbitol and mannitol, and inorganic salts such as sodium chloride. Stabilizers refer to a broad category of excipients which can range in function from a bulking agent to an additive which solubilizes the therapeutic agent or helps to prevent denaturation or adherence to the container wall. Typical stabilizers can be polyhydric sugar alcohols (enumerated above); amino acids such as arginine, lysine, glycine, glutamine, asparagine, histidine, alanine, ornithine, L-leucine, 2-phenylalanine, glutamic acid, threonine, etc., organic sugars or sugar alcohols, such as lactose, trehalose, stachyose, mannitol, sorbitol, xylitol, ribitol, myoinisitol, galactitol, glycerol and the like, including cyclitols such as inositol; polyethylene glycol; amino acid polymers; sulfur containing reducing agents, such as glutathione, thioctic acid, sodium thioglycolate, thioglycerol, a-monothioglycerol and sodium thio sulfate; low molecular weight polypeptides (e.g., peptides of 10 residues or fewer); proteins such as human serum albumin, bovine serum albumin, gelatin or immunoglobulins; hydrophylic polymers, such as polyvinylpyrrolidone monosaccharides, such as xylose, mannose, fructose, glucose; disaccharides such as lactose, maltose, sucrose and trisaccharides such as raffinose; and polysaccharides such as dextran. Stabilizers can be present in the range from 0.1 to 10,000 weights per part of weight polypeptide of the invention. Non-ionic surfactants or detergents (also known as “wetting agents”) can be added to help solubilize the therapeutic agent as well as to protect the therapeutic protein against agitation-induced aggregation, which also permits the formulation to be exposed to shear surface stressed without causing denaturation of the protein. Suitable non-ionic surfactants include polysorbates (20, 80, etc.), poloxamers (184, 188 etc.), Pluronic polyols, polyoxyethylene sorbitan monoethers (TWEEN®-20, TWEEN®-80, etc.). Non-ionic surfactants can be present in a range of about 0.05 mg/ml to about 1.0 mg/ml, or in a range of about 0.05 mg/ml to about 0.2 mg/ml.

Additional miscellaneous excipients include bulking agents (e.g., starch), chelating agents (e.g., EDTA), antioxidants (e.g., ascorbic acid, methionine, vitamin E), and cosolvents.

Freeze-drying or lyophilization, unless otherwise indicated by the context in which it appears, shall be used to denote a drying process in which a solution of materials (i.e. an active pharmaceutical ingredient and various formulation additives or “excipients”) is converted into a solid. A typical freeze-drying process consists of three stages, “freezing”, “primary drying” and “secondary drying”. In the freezing stage almost all contained water is converted into ice and solutes into solids (crystalline or amorphous). In the primary drying stage the ice is removed from the product by direct sublimation which is achieved by maintaining a favorable pressure gradient between the water molecules (ice) and the surrounding atmosphere. In the secondary drying stage residual moisture is removed from the product by desorption.

If concentrations (w/v) are given for freeze-dried compositions they refer to the volume directly prior to freeze-drying.

Unless otherwise noted, percentage terms express weight/weight percentages and temperatures are in the Celsius scale.

Ratios

As described in more detail below, the polypeptide of the invention may be a monomer, a dimer, or a mixture thereof. Any molar ratios according to the invention refer to a ratio of the molar concentration of the dimer of the polypeptide of the invention, whether actually present as homodimer or heterodimer.

Any ratios of the polypeptide of the invention over FVIII in this application refer to the amount of dimer (in mole) comprised in the polypeptide of the invention, which is preferably present as a heterodimer, divided by the amount of FVIII (in mole), unless indicated otherwise. By way of non-limiting example the co-formulation of 100 μM of heterodimeric polypeptide of the invention, consisting of 200 μM monomeric subunits, with 1 μM of FVIII means a ratio of 100.

The molar ratio of the polypeptide of the invention to FVIII is at least 1, preferably at least 2, more preferably at least 4, or at least 10, or at least 20, or at least 25, or at least 50, or greater than 50, or at least 100, or at least 150, or at least 200, or at least 250, or at least 300, or at least 350, or at least 400, or at least 450, or at least 500, or at least 1,000, or at least 1,500, or at least 2,500, or at least 4,000 or up to 5,000. The molar ratio of the polypeptide of the invention to FVIII may according to certain embodiments not exceed a ratio of 5,000, a ratio of 2,500, a ratio of 1,250 or a ratio of 1,000.

The molar ratio of the polypeptide of the invention to FVIII may range from about 1 to 5,000, or from 2 to 2,500, or from 4 to 2,000, or from 10 to 1,500, or from 25 to 1,000, or from 50 to 500. Preferably, the molar ratio of the polypeptide of the invention to FVIII ranges from 1 to 1,250, or from 2 to 1,000, or from 4 to 750, or from 10 to 500.

The above ratios refer to the ratios to be administered in the course of a combination treatment, or to the ratio of both active ingredients in a pharmaceutical composition.

The nucleotide and amino acid sequences shown in the sequence listing are summarized in the Table 1.

TABLE 1:

| SEQ ID NO: | Remarks |
|---|---|
| 1 | cloning vector: pMARES-neo (MCS) |
| 2 | DNA sequence encoding hygroR |
| 3 | Amino acid sequence of D1D2D\grave{ }D3-HSA-TER119scFv (ID 3260) |
| 4 | DNA sequence encoding D1D2D\grave{ }D3-HSA-TER119scFv (ID 3260) |
| 5 | Amino acid sequence of D1D2D\grave{ }D3 8His-tag (ID 3748) |
| 6 | DNA sequence encoding D1D2D\grave{ }D3 8His-tag (ID 3748) |
| 7 | Amino acid sequence of D1D2D\grave{ }D3(EYA)-HSA-TER119scFv (ID 3794) |
| 8 | DNA sequence encoding D1D2D\grave{ }D3(EYA)-HSA-TER119scFv (ID 3794) |
| 9 | Amino acid sequence of D1D2D\grave{ }D3(EYA) 8His-tag (ID 3795) |
| 10 | DNA sequence encoding D1D2D\grave{ }D3(EYA) 8His-tag (ID 3795) |
| 11 | Amino acid sequence of Furin (ID 863) |
| 12 | DNA sequence encoding Furin (ID 863) |
| 13 | Amino acid sequence of D1D2D\grave{ }D3-HSA (CSL626) |
| 14 | Amino acid sequence of D1D2D\grave{ }D3(EYA)-HSA (CSL629) |
| 15 | Amino acid sequence of secreted rVIII-SingleChain (CSL627) |
| 16 | Amino acid sequence of anti-TER119 single chain fragment variable |
| 17 | DNA sequence encoding the pre-pro form of human native VWF |
| 18 | Amino acid sequence encoded by SEQ ID NO: 17 |
| 19 | Amino acid sequence of mature human serum albumin |
| 20 | Amino acid sequence of human native FVIII |
| 21 | Amino acid sequence of a VHH nanobody designated IH4, disclosed in |
| | WO 2014/135528 A1 |
| 22 | DNA sequence encoding scFv 1C3, obtainable from hybridoma G26.4.1C3/86 |
| | [RAT 1C3/86] (ATCC ® HB-9893 ™) |
| 23 | Amino acid sequence encoded by SEQ ID NO: 22 |

Certain embodiments of the invention will now be described with reference to the following examples which are intended for the purpose of illustration only and are not intended to limit the scope of the generality hereinbefore described.

### Examples

Results

D′D3-TER119 Dimer Cross-Links Murine RBCs In Vitro

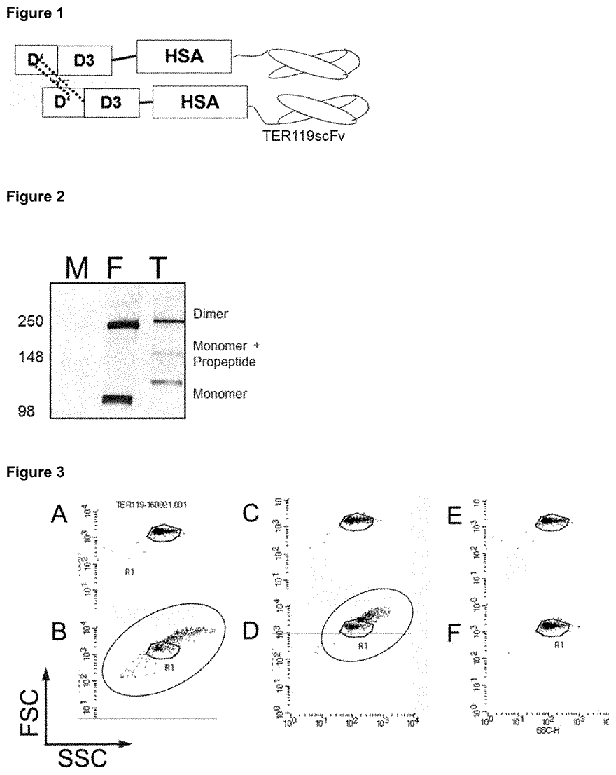

A recombinant VWF D′D3 albumin fusion protein (rD′D3-FP) has been developed previously to extend the half-life of co-administered FVIII for the treatment of hemophilia A (ref. paper, A245). We thought that a fusion of D′D3-FP with TER119scFv (TER119-specific single chain Fv; antibody fragment with specificity for murine glycophorin A) may act as a mediator targeting FVIII to RBC through its bispecific nature in situ. A rD′D3-FP fused with TER119scFv (D′D3-FP-TER119scFv, SEQ ID NO:3) was stably expressed in line with VWF propeptide (D1D2) which is essential for efficient dimerization of D′D3 domains (FIG. 1). The CHO expression cell line secreted monomers and homodimers of D′D3-FP-TER119scFv as expected (FIG. 2). Both species were purified separately. FVIII (rVIII-SingleChain, CSL Behring, Marburg, Germany) could indeed be detected on the surface of RBCs titrating both, monomer and homodimer of D′D3-FP-TER119scFv, in presence of 5 μg/ml FVIII in vitro. RBCs were stained efficiently by polyclonal anti-FVIII-FITC using the homodimer, but not the monomer (data not shown). However, we observed that RBCs formed visible aggregates using the homodimer, but not the monomer, at physiologically relevant concentrations (>5 μg/ml) in vitro. Surprisingly, the monomer cross-linked RBC only in the presence of a detection antibody against human albumin (FIG. 3). We considered this finding as a potential safety risk for in vivo applications.

It was previously demonstrated that FVIII is bound efficiently by D′D3 dimers, but not by monomeric D′D3. Monomeric D′D3 has significantly decreased affinity towards its natural ligand, FVIII, compared with D′D3 dimer (WO 2018/087271 A1). FVIII-RBCs interaction would further be inhibited by endogenously circulating VWF in an in vivo scenario. We have previously demonstrated that dimeric D′D3-FP extends the half-life of FVIII only when it is administered at doses which compete and exceed the molar concentration of endogenous VWF in plasma. A D′D3 monomer would have to be injected at significant higher doses than D′D3 dimer because of its lower affinity towards FVIII. Thus, overcoming the problem of cross-linked RBCs through developing a monomeric D′D3-FP-TER119scFv was not an option for us. A dimerized D′D3 as subunit in a bispecific molecule was preferable.

D′133-TER119 Heterodimer Facilitates FVIII Binding to Murine RBCs without Crosslinking In Vitro

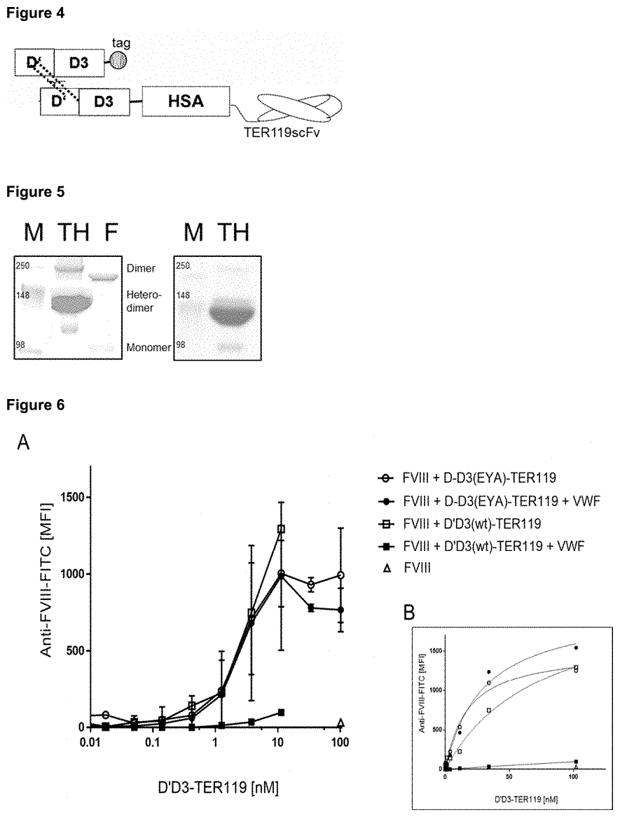

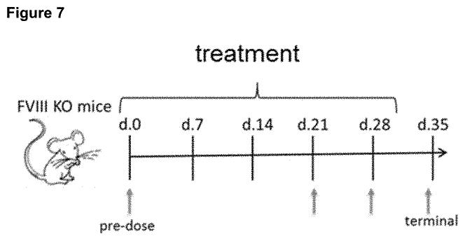

Surprisingly, D′D3-FP-TER119scFv (SEQ ID NO:3) and D′D3 (without albumin and TER119scFv fusion partners, SEQ ID NO:5) co-expressed in a stably transfected cell line were secreted as heterodimers which could be purified using two affinity steps against human albumin and a c-terminal tag of the non-fused D′D3 (FIG. 4 and FIG. 5). Same expression strategy was applied using a high affinity variant of D′D3_{EYA}-FP-TER119scFv (SEQ ID NO:7) and D′D3_{EYA} (SEQ ID NO:9) previously published, known as “D′D3 EYA” or CSL629 (WO 2017/117630 A1, WO 2017/117631 A1; SEQ ID NO:14). FIG. 6 illustrates that D′D3-FP-TER119scFv, both as wildtype (WT) and the high affinity variant (EYA), facilitates binding of human FVIII in a concentration-dependent manner to murine RBCs in vitro. Briefly, a fixed concentration of rVIII-SingleChain (12.5 IU/mL, approx. 6 nM, CSL Behring Marburg) was incubated with increasing concentration of both purified heterodimer constructs, D′D3_{WT}-TER119 and D′D3_{EYA}-TER119, before washed murine RBCs from 1:100 diluted fresh mouse blood were added and analyzed. FVIII on the surface of murine RBCs was specifically detected by polyclonal anti human FVIII FITC-labelled antibodies. Importantly, the bispecific, but monovalent, design of the D′D3-TER119 heterodimer did not show aggregation or crosslinking of murine RBCs, neither visible nor based on SSC/FSC (single cell) analysis. Human plasma derived VWF (12.5 IU/ml, approx. 250 nM, CSL Behring, Marburg) was added as indicated. Thus, as per plasma unit definition the molar ratio of FVIII and VWF was assumed to be 1 to 35 and physiologically relevant. Selecting equal and fixed standard human plasma unit concentrations of FVIII and VWF (12.5 IU/ml) while titrating both D′D3-TER119 heterodimer constructs, the competition of both, D′D3-TER119 and endogenous VWF, for their common ligand, FVIII, was simulated in vitro. Interestingly, VWF inhibits FVIII binding to murine RBCs using D′D3_{WT}-TER119, but not D′D3_{EYA}-TER119. That is because human VWF and D′D3_{WT}-TER119 have the same amino acid sequence of their FVIII binding subunit, D′D3, and so equal affinities towards FVIII (WO 2017/117630 A1, WO 2017/117631 A1). Where D′D3_{WT}-TER119 does not facilitate binding of FVIII towards murine RBCs in presence of human VWF, D′D3_{EYA}-TER119 is capable to target FVIII even at molarities lower than those of VWF. This observation can be explained with the 30-fold higher affinity of the D′D3(EYA)-FP (CSL629) variant compared with wild type D′D3-FP (CSL626) or VWF towards FVIII (WO 2017/117630 A1, WO 2017/117631 A1).

TABLE 2
Calculated KM for FVIII binding to murine RBCs upon
D′D3-TER119 titration. Michaelis-Menten-Kinetic

| | | in vitro study (a) | | in vitro study (b) | |
|---|---|---|---|---|---|
| Construct | Batch | | +VWF | | +VWF |
| D′D3_{EYA}-TER119 | #1 | 17.8 nM ± | 32.4 nM ± | 3.7 nM ± | 3.6 nM ± |
| | | 0.5% | 0.7% | 5.7% | 0.2% |
| | #2 | | | 9.3 nM ± | 9.9 nM ± |
| | | | | 8.3% | 2.0% |
| D′D3_{WR}-TER119 | | 77.6 nM ± | n/a | | |
| | | 1.2% | | | |

The apparent KM of the binding kinetic of FVIII bound to D′D3_{EYA}-TER119 towards murine RBCs were calculated assuming a Michaelis-Menten kinetic (FIG. 6B). A summary of calculated KM values of two independent in vitro experiments is provided in Table 2. One in vitro experiment suggested that the presence of VWF increases the KM of FVIII binding to RBCs using D′D3_{EYA}-TER119 (32.4 nM±0.7%) compared with data where no VWF was added (17.8 nM±0.5%). This observation was not confirmed looking at the qualitative trends (FIG. 6A). Further, a repeat of the experiment using two batches of D′D3_{EYA}-TER119 demonstrated that there was no significant difference in KM neither in absence nor in presence of VWF (3.7 nM±5.7% and 3.6 nM±0.2%). We observed that a second batch of D′D3_{EYA}-TER119 had an increased KM, both with or without VWF (9.3 nM±8.3% and 9.9 nM±2.0%) compared to a first batch material. However, the trends of the titration curves (up to 102 nM) did not represent that observation.

The titration of D′D3_{WT}-TER119 shows an increased KM (25.6 nM±4%) compared with KM of D′D3_{EYA}-TER119 without VWF, but again the trend in FIG. 6A did not reflect this observation. Interestingly, the KM of D′D3_{WT}-TER119 with VWF could not be calculated because FVIII binding on RBCs was only weakly detected at high concentrations of D′D3_{WT}-TER119 (between 4 and 12.5 nM), where D′D3_{EYA}-TER119 with VWF already demonstrated maximal binding. Since a fixed excess molar VWF concentration of approx. 250 nM was used in these in vitro experiments competing with D′D3-TER119 variants towards the common binding partner FVIII, our flow cytometry binding studies show clearly a beneficial effect of targeting FVIII to RBC using the high affinity variant of the bispecific heterodimer, D′D3_{EYA}-TER119.

Reduction of FVIII Antibodies in FVIII Ko Mice Treated with D′D3-TER119 Heterodimer

Weekly dosing of rFVIII is known to lead to anti-drug antibody (ADA) formation, typically shown to be inhibitory antibodies (measured by Bethesda units) in FVIII k.o. mice. In this example, the effect of D′D3_{EYA}-TER119 heterodimer on development of ADA against a rFVIII, rVIII-SingleChain, was investigated in comparison to a non-erythrocyte binding control, D′D3(EYA)-FP.

Recombinant FVIII (rVIII-SingleChain, 200 IU/kg, approx. 16 μg/kg) co-administered with 100 μg/ml D′D3(EYA)-FP (CSL629), previously published as CSL629, was reported to extend the half-life of infused FVIII in vivo (WO 2018/087271 A1). One explanation of the mode of action has been discussed: the molar ratio of FVIII and its high affinity chaperon, D′D3(EYA)-FP (CSL629), of at least 1 to 4 was sufficient to capture the majority of injected FVIII molecules in circulation. Thus, only a minor portion of injected FVIII bound to endogenous VWF, and not to CSL629, which, however, was shown to be still capable to rescue FVIII ko mice in bleeding models (data not shown). We applied the same molar ratio (1 to 4) but at approx. ten-fold higher doses in vivo to induce tolerance towards FVIII. For initial dosing regimen it was also considered that less than 1 mg/kg of human, heterologous protein, was injected to avoid anaphylaxis.

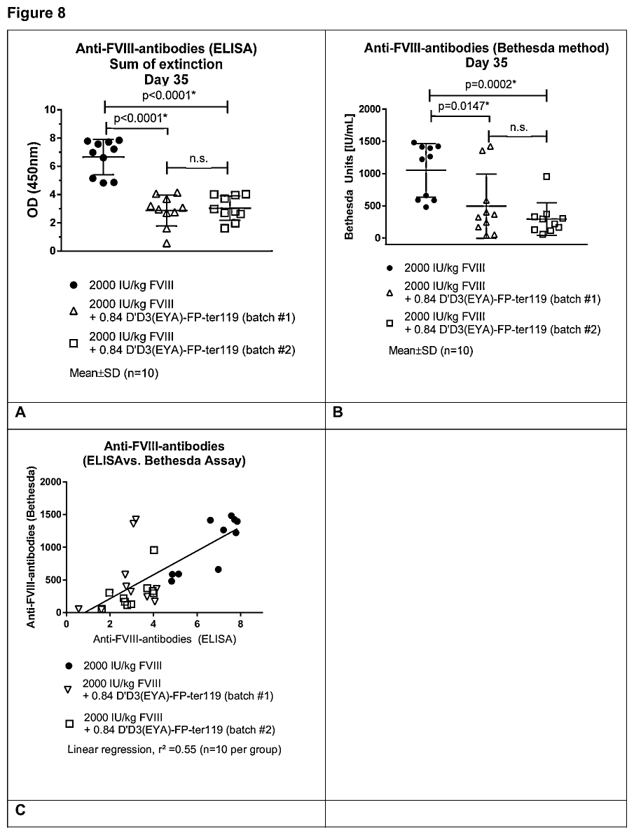

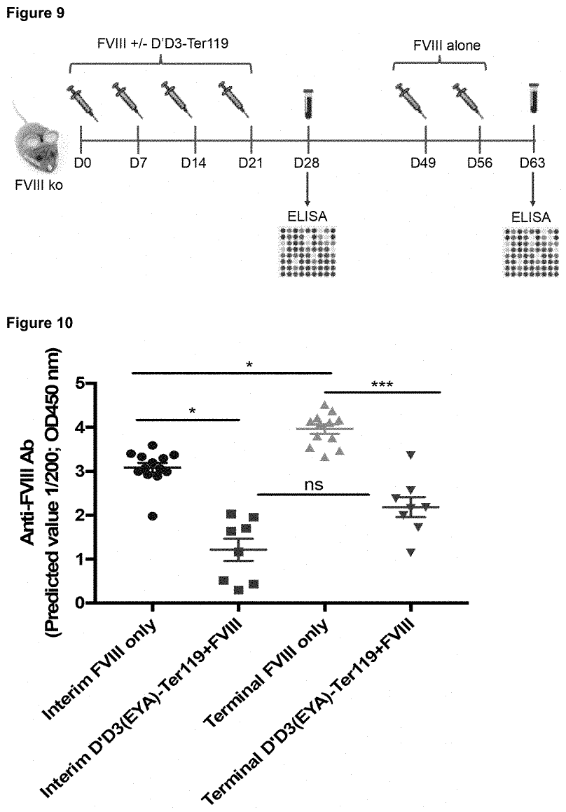

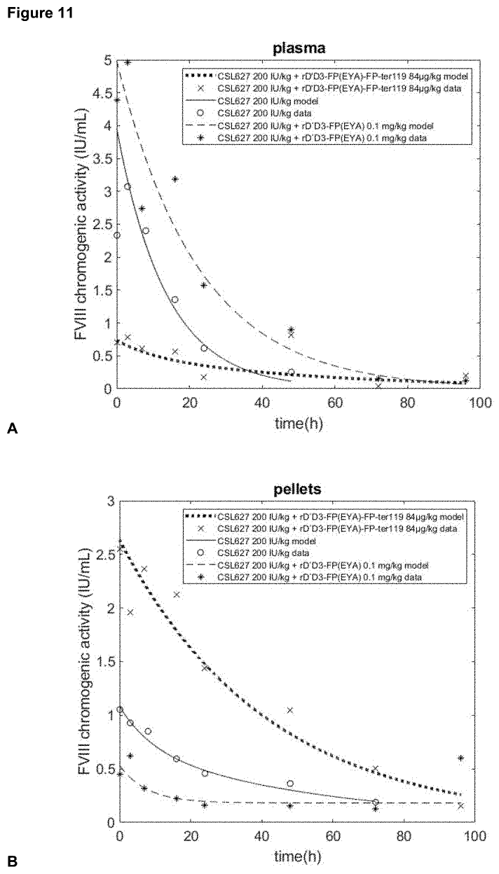

Administration of rVIII-SingleChain (2000 IU/kg) with or without D′D3_{EYA}-TER119 heterodimer (840 μg/kg) to n=10 FVIII k.o. mice per group on days 0, 7, 14, 21 and 28 (treatment regimen see FIG. 7) led to generation of anti-FVIII-ADA and inhibitory antibodies neutralizing FVIII activity. Anti-FVIII ADA values were given as sum of extinctions and are shown as individual values and mean±SD (FIG. 8A). rVIII-SingleChain at a dose of 2000 IU/kg given alone to FVIII k.o. mice led to generation of ADA on day 35 of 6.61±1.14. Co-administration of D′D3_{EYA}-TER119 heterodimer at a dose of 840 μg/kg using two different batches significantly reduced the sum of extinctions to 2.88±1.04 (batch 1) and 3.04±0.82 (batch 2). There was no significant difference between the two co-administered D′D3_{EYA}-TER119 heterodimer batches (FIG. 8A).

The generated ADA against FVIII were shown to be inhibitory as quantified by Bethesda units, as can be seen when plotted for each individual animal and mean±SD (FIG. 8B), which shows generally the same response. Additionally, each individual ADA value (sum of extinction) was compared with the inhibitory potential quantified in Bethesda Units, and a linear regression was calculated over all values with an r² value of 0.55 (FIG. 8C), thereby supporting a direct correlation between anti-FVIII ADA sum of extinction and their inhibitory potential measured as Bethesda Units.

In conclusion, two independent batches of D′D3_{EYA}-TER119 heterodimer reduced the immunogenic potential of rVIII-SingleChain in FVIII k.o. mice.

D′D3EYA-TER119 Induces Long-Term Tolerance Towards FVIII in FVIII k.o. Mice

We were also interested whether D′D3_{EYA}-TER119 heterodimer is efficacious to induce long-term tolerance towards FVIII. Therefore, FVIII knockout mice were injected intravenously (i.v.) once a week for four weeks with 1700 IU/kg FVIII (AFSTYLA®, approx. 136 μg/kg CSL Behring, Marburg, Germany) per mouse with or without D′D3_{EYA}-TER119 (672 μg/kg, an approx. four-fold molar excess compared to FVIII). An interim bleed was carried out one week after the fourth injection and plasma was analyzed by ELISA for anti-FVIII antibodies. After additional four weeks, mice were re-challenged with two weekly injections of FVIII alone (120 IU/kg, i.v.). Seven days after the last injection with FVIII, mice were euthanized for terminal plasma collection and plasma was analyzed by ELISA for anti-FVIII antibodies. Surprisingly, treatment with D′D3_{EYA}-TER119 heterodimer in combination with FVIII significantly reduced antibody formation against FVIII and anti-FVIII antibody levels did not significantly increase even after re-challenge.

TABLE 3
PK parameters of FVIII levels in plasma in FVIII k.o. mice.

| | | | | | CSL627 200 IU/kg + | |
|---|---|---|---|---|---|---|
| PK parameters | | | CSL627 200 IU/kg + | | rD\acute{ }D3-FP(EYA)-FP- | |
| of plasma FVIII | CSL627 | | rD\acute{ }D3-FP(EYA) | | ter119 (Heterodimer) | |
| levels in FVIII | 200 IU/kg | | 0.1 mg/kg | | 84 μg/kg | |
| ko mice. | Mean | SD | Mean | SD | Mean | SD |
| AUC_{_{—}}_{inf} (h*IU/mL) | 53.5 | 0.5 | 112.2 | 13.1 | 30.2 | 5.3 |
| AUC_{_{—}}ₗₐₛₜ (h*IU/mL) | 52.0 | 0.5 | 110.6 | 13.1 | 25.5 | 3.8 |
| % extrapol | 2.9 | 0.2 | 1.4 | 0.6 | 15.7 | 8.2 |
| Clearance (mL/kg/h) | 3.7 | <0.1 | 1.8 | 0.2 | 6.6 | 4.1 |
| MRT (h) | 13.5 | 0.2 | 22.5 | 2.0 | 50.8 | 16.3 |
| Half-Life_term (h) | 9.4 | 0.2 | 15.6 | 1.6 | 37.4 | 13.2 |
| V_{c} (mL/kg) | 50.6 | 1.2 | 40.1 | 9.7 | 270.8 | 27.5 |
| Vₛₛ (mL/kg) | 50.6 | 1.2 | 40.1 | 7.1 | 336.6 | 86.6 |
| V_{z} (mL/kg) | 50.6 | 1.2 | 40.1 | 8.7 | 357.7 | 111.3 |
| Cₘₐₓ (IU/mL) | 4.0 | <0.1 | 5.0 | <0.1 | 0.7 | <0.1 |
| IVR (%) | 79.1 | <0.1 | 99.7 | <0.1 | 14.8 | <0.1 |

TABLE 4
PK parameters of FVIII levels in pellet in FVIII k.o. mice.

| | | | | | CSL627 200 IU/kg + | |
|---|---|---|---|---|---|---|
| | | | CSL627 200 IU/kg + | | rD\acute{ }D3-FP(EYA)-FP- | |
| | CSL627 | | rD\acute{ }D3-FP(EYA) | | ter119 (Heterodimer) | |
| | 200 IU/kg | | 0.1 mg/kg | | 84 μg/kg | |
| | Mean | SD | Mean | SD | Mean | SD |
| AUC_{_{—}}_{inf} (h*IU/mL) | 43.2 | 5.6 | 67.1 | 15.5 | 109.1 | 9.3 |
| AUC_{_{—}}ₗₐₛₜ (h*IU/mL) | 32.0 | 4.2 | 14.3 | 0.5 | 98.4 | 7.4 |
| % extrapol | 25.8 | 7.6 | 78.7 | 13.8 | 9.9 | 2.3 |
| Clearance (mL/kg/h) | 4.6 | 3.0 | 3.0 | 1.9 | 1.8 | 0.2 |
| MRT (h) | 52.9 | 13.9 | 357.2 | 99.9 | 41.4 | 4.1 |
| Half-Life_term (h) | 39.4 | 11.5 | 259.4 | 72.1 | 28.7 | 2.9 |
| V_{c} (mL/kg) | 184.8 | 81.9 | 386.0 | 82.1 | 75.9 | 7.8 |
| Vₛₛ (mL/kg) | 245.1 | 54.9 | 1064.1 | 148.7 | 75.9 | 6.9 |
| V_{z} (mL/kg) | 263.1 | 61.9 | 1114.7 | 155.2 | 75.9 | 7.2 |
| Cₘₐₓ (IU/mL) | 1.1 | 0.0 | 0.5 | 0.0 | 2.6 | 0.0 |
| IVR (%) | 21.6 | 0.0 | 10.1 | 0.0 | 52.7 | 0.0 |

na = not applicable

1. A polypeptide comprising (i) a VWF moiety and (ii) an erythrocyte-binding moiety, wherein said polypeptide is capable of binding to blood coagulation factor VIII (FVIII).

2. The polypeptide of claim 1, wherein said VWF moiety is capable of binding to FVIII.

3. The polypeptide of claim 1, wherein said VWF moiety is a truncated VWF comprising the D′D3 domain of a VWF.

4. The polypeptide of claim 1, wherein said VWF moiety comprises at least one amino acid substitution as compared to the amino acid sequence of wild-type VWF as shown in SEQ ID NO:18.

5. The polypeptide of claim 4, wherein said at least one amino acid substitution is selected from the group of combinations consisting of S764G/S766Y, S764P/S766I, S764P/S766M, S764V/S766Y, S764E/S766Y, S764Y/S766Y, S764L/S766Y, S764P/S766W, S766W/S806A, S766Y/P769K, S766Y/P769N, S766Y/P769R, S764P/S766L, and S764E/S766Y/V1083A, referring to the sequence of SEQ ID NO:18 with regard to the amino acid numbering.

6. The polypeptide of claim 1, wherein said polypeptide is a dimer.

7. The polypeptide of claim 1, wherein said polypeptide is a heterodimer.

8. The polypeptide of claim 7, wherein the heterodimer comprises a first subunit and a second subunit, wherein the first subunit comprises the VWF moiety and the erythrocyte-binding moiety, the second subunit comprises a second VWF moiety, and the second subunit does not comprise an erythrocyte-binding moiety.

9. The polypeptide of claim 1, wherein said erythrocyte-binding moiety is capable of binding to a membrane protein on an erythrocyte.

10. The polypeptide of claim 1, wherein said erythrocyte-binding moiety is selected from the group consisting of a peptide ligand, an antibody, an antibody fragment, and a single chain antigen binding domain (scFv).

11. A pharmaceutical composition comprising the polypeptide of claim 1, and optionally a pharmaceutically acceptable carrier, diluent, or excipient.

12.-17. (canceled)

18. A nucleic acid encoding the polypeptide of claim 1.

19. A plasmid or vector comprising the nucleic acid of claim 18.

20. A host cell comprising the plasmid or vector of claim 19.

21. A method of producing a polypeptide comprising a VWF and an erythrocyte-binding moiety, comprising (i) culturing the host cell of claim 20 under conditions such that the polypeptide comprising the VWF and the erythrocyte-binding moiety are expressed; and (ii) optionally recovering the polypeptide comprising the VWF and the erythrocyte-binding moiety from the host cells or from the culture medium.

22. A method of increasing the half-life of FVIII in a subject in need thereof, comprising administering to the subject an effective amount of the polypeptide of claim 1.

23. A method of reducing or preventing inhibitor formation in a subject being treated with FVIII, said method comprising administering to said subject an effective amount of the polypeptide of claim 1.

24. A method of increasing the half-life of FVIII in a subject in need thereof, comprising administering to the subject an effective amount of the pharmaceutical composition of claim 11.

25. A method of reducing or preventing inhibitor formation in a subject being treated with FVIII, said method comprising administering to said subject an effective amount of the pharmaceutical composition of claim 11.

26. A method of treating a blood coagulation disorder in a patient in need thereof comprising administering to the patient a therapeutically effective amount of the polypeptide of claim 1.

27. A method of treating a blood coagulation disorder in a patient in need thereof comprising administering to the patient a therapeutically effective amount of the pharmaceutical composition of claim 11.

28. The method of claim 26, further comprising administering a therapeutically effective amount of FVIII.

29. The method of claim 27, further comprising administering a therapeutically effective amount of FVIII.

30. A method of increasing the half-life of FVIII in a patient in need thereof, comprising administering to the patient an effective amount of the polypeptide of claim 1.

31. A method of increasing the half-life of FVIII in a patient in need thereof, comprising administering to the patient an effective amount of the pharmaceutical composition of claim 11.
